(19) United States
(12) Patent Application Publication
(10) Pub. No.: US 2009/0215179 A1
(43) Pub. Date: Aug. 27, 2009

(54) Transgenically preventing establishment and spread of transgenic algae in natural ecosystems

(75) Inventors: Jonathan Gressel, Rehovot (IL); Shai Ufaz, Givat-Ada (IL); Ofra Shen, Rehovot (IL); Doron Eisenstadt, Haifa (IL)

Correspondence Address: DODDS & ASSOCIATES, 1707 N STREET NW, WASHINGTON, DC, 20036, US

(73) Assignee: TransAlgae, Rehovot (IL)

(21) Appl. No.: 12/322,686

(22) Filed: Feb. 5, 2009

Related U.S. Application Data
(63) Continuation-in-part of application No. 10/774,388, filed on Feb. 10, 2004.
Application No. 10/774,388 is a continuation-in-part of application No. 09/889,737, filed on Jul. 20, 2001, now abandoned.

(51) Int. Cl. C12N 15/74

(52) U.S. Cl. 435/471; 435/320.1

(57) ABSTRACT

Genetic mechanisms for mitigating the effects of introgression of a genetically engineered genetic trait of cultivated algae or cyanobacteria to its wild type or to an undesirable, interbreeding related species. as well as preventing the establishment of the transgenic algae or cyanobacteria in natural ecosystems.

This application is a Continuation-in-Part application, of U.S. application Ser. No. 10/774,388 which is Continuation-in-Part of application Ser. No. 09/889,737.

### FIELD AND BACKGROUND OF THE INVENTION

The present invention relates to a genetic mechanism for preventing the establishment of transgenic algae and cyanobacteria in natural ecosystems should they be released from enclosed cultivation.

Algae and cyanobacteria have recently attracted much interest as biofactories for production of foods, bioactive compounds and biofuels. Since algae and cyanobacteria need sunlight, carbon-dioxide, and water for growth, they can be cultivated in open or enclosed water bodies. These systems are vulnerable to being contaminated by other algal species and cyanobacteria. Similarly, the cultivated algae may escape outside the cultivation. This may become a serious concern when the cultivated cells are transgenically modified.

The release of organisms containing introgressed genetically engineered genetic traits may have negative environmental impacts and be of regulatory concern, and thus it is imperative that algae and cyanobacteria containing transgenic traits not establish outside of their place of cultivation. Various methods have been described for prevention of transgene flow in crop plants. We have previously described a method to mitigate the establishment of transgenes from crops that might disperse to the wild, either through seed or through gene introgression into weedy or wild relatives of crops, described in U.S. patent application Ser. Nos. 09/889,737 and 10/774,388 of which this application is a continuation in part. Both of the previous applications are incorporated herein by reference.

While the major type of introgression from transgenic crops is sexual interspecific genetic gene flow, and in some cases sexual gene flow to related species, in the case of algae and cyanobacteria it is mainly that they themselves will establish and propagate asexually, as sexual exchanges are quite rare with most algal and cyanobacterial species. Still, cyanobacteria can be subject to horizontal gene flow through phages and possibly by conjugation. Horizontal gene flow is rare in eukaryotic organisms including algae, but conjugation-like processes have been confirmed, intra-specifically in the laboratory by protoplast fusion (Sivan and Arad, 1998). What can occur in the laboratory at high frequency intra-specifically, can happen at much lower frequencies in nature, posing a finite risk, possibly even between related species. Thus, we here extend the concept described for higher plants in the above mentioned patent application to algae and cyanobacteria; tandemly combine a gene that is needed in the transgenic algae or cyanobacteria and poses a risk in natural ecosystems, with another gene that is either useful or neutral to the cultivated algae or cyanobacteria, but would be deleterious to the organisms in natural ecosystems such that there is a net fitness disadvantage. Because of the tandem construct, they remain genetically linked through asexual or sexual propagation, or gene flow. In cases where there is no sexual or asexual recombination, the genes may be introduced separately.

Thus, a series of genes that have either a neutral or desirable effect on the algae and cyanobacteria in cultivation but will prevent competition and establishment in the environment are genetically engineered into the algae and cyanobacteria. These would override any selective advantage derived from other possible transgenes that might provide a modicum of advantage in natural ecosystems.

### Rapid Domestication of Algae and Cyanobacteria as Crops for Industrial and Agricultural Uses by Transgenesis

Higher plants have been domesticated as crops since prehistory, by farmers who selected against a large number of traits that were valuable for wild species, but undesirable in agronomic practice. These differences between wild species and crops were further accentuated by selective breeding, and even more so by genetic engineering, which allowed introducing traits that were non-existent in the gene pool of the species, genus, family, or kingdom of the crop.

Algae and cyanobacteria have only recently been considered for wide scale cultivation with domestication limited to mainly selection of organisms, occasionally with selection of strains or mutants with desired traits. Unlike crops, millennia of efforts have not been invested in their domestication, and in many cases the traits needed do not exist within the species. Genetic engineering allows one to rapidly fill the void of needed traits for rapid domestication (e.g. Gressel, 2008a). Indeed, large scale cultivation of algae has been plagued by problems that are analogous to agricultural production of crops (Gressel 2008b; Sheehan et al., 2004). These problems include contamination by other algae and cyanobacteria (analogous to weeds in crops), fungi, bacteria, viruses (analogous to pathogens of crops), zooplankton (analogous to arthropod pests of crops), low productivity, and especially of desired traits (dealt with in crops by breeding for millennia). With crops, the analogous problems with cultivation, light penetration, light use efficiency, heating, mineral nutrition, harvesting have been dealt with by breeding coupled with development of novel cultivation procedures, and are being continued with the added tools of genetic engineering, which allows bringing in traits not available in the organism's genome.

Introgression of genetically engineered traits: Needed traits could be artificially forced horizontally into the algae and cyanobacteria by genetic engineering to enhance cost-effectiveness (higher yields, new products, resistances to contaminations, adaptability to cultivation with high levels of light and carbon dioxide not presently occurring in their natural ecosystems). Detractors of both the process of genetic engineering and its products have raised the possibilities that the engineered algae and cyanobacteria would become uncontrollable problems if there was an inadvertent leak or spill from such cultivation into natural ecosystems. The benefits that accrue from cultivating transgenic algae and cyanobacteria, with their much higher primary productivity than terrestrial crops, could have great benefits to humanity by providing equivalent products on far less land area than conventional agriculture, often using seawater instead of potable fresh water, with far less fertilizer, and without fertilizer or pesticide run-off, allowing removing of agricultural land from production and putting the land to more environmentally sound use.

Risk analysis and risk mitigation: Tomes have been written on how to assess the risks of introgression in crops—some with continuing generalizations and some discussing how and why this assessment must be undertaken on a case by case basis (Regal, 1994; Keeler et al., 1996; Kareiva et al., 1996; de Kathen, 1998; Williamson, 1993; Timmons et al., 1996; Kjellsson et al., 1998; Sindel, 1997; Gressel and Rotteveel, 2000, Galun and Breman, 1997; Krimsky and Wrubel, 1996). There have been no published assessments on risks from transgenic algae, but it is clear that some traits being engineered into algae or cyanobacteria could give them a selective advantage in competing with other algae and cyanobacteria in natural ecosystems. This would include genes encoding enzymes that enhance utilization of fixed carbon and its sequestration into stored products such as fructose-1,6-bisphosphate aldolase (ALD) and triose phosphate isomerase (TPI) as well as phosphoribosylphosphate synthetase (Ma et al. 2007; Kang, et al., 2005). Transgenes encoding resistance to viruses or phages could easily be predicted to supply a competitive advantage to the recipient. Similarly, genes for herbicide resistance could provide a selective advantage in the few areas where herbicides are used (typically in freshwater, not marine habitats). It might be harder to predict the competitive advantages of other genes such as enhanced or modified lipid, amino acid, protein or carbohydrate contents, but precaution might prevent the utilization of such genes, unless there are mechanisms in place to prevent their establishment in natural ecosystems, should they escape cultivation.

Two general approaches deal with the problems of transgene flow from crops that could be considered for use in algae and cyanobacteria: containment of the transgenes within the transgenic crop, and transgenic mitigation of the effects of the primary transgenic trait should it escape and move to an undesired target. Many containment efforts have depended upon physical containment (see Physical Gene Flow Barriers, page 61; in: Environmental issue report, No. 28, European Environmental Agency Publication No. 28, 2002), but it is breaches in physical containment that require precaution. Of the biological means such as apomixis, cleistogamy, male sterility and plastid transformation proposed for crops (see Biological Gene Flow Barriers, pages 60 and 61; in: Environmental issue report, No. 28, European Environmental Agency Publication No. 28, 2002, and Daniell H, Nature Biotechnology, 2002; 20:581-86) and the recoverable block of function concept introduced in U.S. Pat. No. 6,849,776 to Kuvshinov et al. or introduction lethal traits under control of inducible promoters (U.S. Pat. No. 5,723,765 to Oliver et al), none would preclude establishment of transgenic algae or cyanobacteria, except for cases where there might be rare sexual transmission. Thus, establishment of transgenic algae and cyanobacteria themselves, not just their introgressed offspring must be mitigated. Unfortunately, discussions of the hazards and risk assessment rarely consider how biotechnologies can be used to mitigate the risk of crop gene establishment in natural ecosystems (see Gressel 2008a as an exception), and there has been no discussion how this might be done for transgenic algae and cyanobacteria.

There is thus a recognized need for, and it would be highly advantageous to have, failsafe anti-establishment, establishment-mitigating mechanisms to reduce the possibility of establishment of algae and cyanobacteria released to natural ecosystems that will also preclude establishment of rare cases where the transgenes interspecifically introgress into other algae or cyanobacteria.

### SUMMARY OF THE INVENTION

According to the present invention a method is provided to obtain transgenic algae or cyanobacteria bearing at least one genetically engineered, commercially desirable genetic trait that is at risk of establishing in natural ecosystems (Table 1) but is tandemly linked to, and co-expressing at least one transgene (mitigating gene) that is desirable in or neutral to the cultivated transgenic algae or cyanobacteria but rendering the transgenic algae or cyanobacteria incapable of establishing by itself or in introgressed offspring in natural ecosystems (Table 2), thereby obtaining a cultivated algae or cyanobacteria capable of mitigating the effects of release of said genetically engineered, commercially desirable genetic trait of the algae or cyanobacteria in natural ecosystems. The sequence encoding the desirable genetic trait and the sequence of the mitigating gene remain genetically linked in the transgenic algae or cyanobacteria according to this invention, because of the introduction of the sequences in tandem. If there is no recombination in the species, the genes need not be tandemly linked.

According to the present invention the transgene that prevents the establishment of the algae or cyanobacteria may be, one or more of the following:

1. a transgene encoding a reduced content of RUBISCO (ribulose 1,5 bis phosphate carboxylase/oxygenase) (such as an antisense or RNAi construct of the small or the large subunit of RUBISCO) which allows normal algae or cyanobacteria growth only at artificially high carbon dioxide concentrations, but not in natural environments;
2. a transgene encoding reduced photosystem 2 antennae size such as tla1, which allows growth only at high light intensities but allows greater packing of cells in commercial production facilities with less light energy wasted as heat; such organisms would not have enough chlorophyll to compete with indigenous organisms in natural ecosystems. In cases where there is no recombination in the species, the gene of choice can be introduced into a mutant strain having a reduced antennae.
3. an antisense or RNAi construct of any of the genes encoding cilia or flagella (or similar motility organ) formation or action such that the transgenic algae or cyanobacteria cannot optimally position themselves based on environmental stimuli. Such movement is required to compete in natural ecosystems, but is unnecessary and utilizes energy in commercial cultivation;
4. a transgenic mutant form of the phytoene desaturase (pds) gene conferring resistance to phytoene desaturase inhibiting herbicides, which synthesizes less beta-carotene. The herbicide resistance allows controlling unwanted species in commercial culture and the less carotene is of little consequence in dense commercial culture but provides photoprotection to organisms in the natural environments and organisms with less beta carotene are less competitive in the natural ecosystems. Similarly, other herbicide resistances can be used;
5. a transgene in the anti-sense or RNAi form encoding one or more of the polymers of the cell walls such that the algae or cyanobacteria has a thinner cell wall. This thinner cell wall is of little consequence in commercial production, and the cell walls are the least commercially valued part of the cell, but organisms with thinner cell walls cannot compete in the variable vicissitudes of environmental conditions in natural ecosystems;
6. a transgene encoding a storage polymer such as inulin, levan or, graminan that the cell cannot later degrade for use as energy when needed, especially if coupled with a transgene in the RNAi or antisense form such as starch phosphorylase, precluding storage as starch. This is desirable in commercial production when the new polymer has a greater value than starch, but renders an organism that cannot mobilize reserves less fit in a natural environment, where it cannot compete with organisms that can mobilize reserves in times of need; or
7. any other transgene that is neutral or beneficial to the algae or cyanobacteria when cultivated commercially, but renders the algae or cyanobacteria unfit to compete in natural ecosystems, overcoming any benefit that may derive from the transgene tandemly bound to it.

Thus a method is provided to obtain a cultivated algae or cyanobacteria having multiple transgenes in tandem, (or in some cases separately introduced), derived from different sources with at least one of the transgenes capable of mitigating the fitness effects preventing stable establishment of at least one genetically engineered, commercially desirable genetic trait of the algae or cyanobacteria in natural ecosystems.

According to yet another aspect of the present invention there is provided a method of obtaining a cultivated algae or cyanobacteria capable of mitigating the effects of self propagation or of asexual or sexual introgression of at least one genetically engineered, commercially desirable genetic trait to an undesirable species related to the cultivated algae or cyanobacteria, the method comprising transforming a population of the cultivated algae or cyanobacteria to express at least one genetically engineered commercially desirable genetic trait in the algae or cyanobacteria under genetic control of at least one genetic control element which is inexpressible by said undesirable interbreeding species related to the cultivated algae or cyanobacteria, thereby obtaining a cultivated algae or cyanobacteria capable of mitigating the effects of self propagation or of introgression of said genetically engineered, commercially desirable genetic trait of the cultivated algae or cyanobacteria to said undesirable species related thereto.

According to a further aspect of the present invention there is provided a genetic construct for genetically modifying a cultivated algae or cyanobacteria to express a genetically engineered, commercially desirable genetic trait while mitigating the effects of establishment of self propagation or sexual or asexual gene introgression of said genetically engineered, commercially desirable genetic trait of the algae or cyanobacteria to an undesirable species related to the cultivated algae or cyanobacteria, the genetic construct comprising a first polynucleotide encoding said genetic trait and at least one additional polynucleotide comprising at least one control element which is expressible by the cultivated algae or cyanobacteria, said control element being inexpressible by said undesirable interbreeding species.

According to yet another aspect of the present invention there is provided a method of obtaining cultivated asexual, non-conjugating algae or cyanobacteria capable of mitigating the effects of self propagation of at least one genetically engineered, commercially desirable genetic trait in natural ecosystems, the method comprising transforming a population of the cultivated algae or cyanobacteria to express at least one genetically engineered, commercially desirable genetic trait into algae or cyanobacteria bearing a natural or induced mutation that acts as a mitigating genetic trait, wherein said mitigating genetic trait is selected such that a self propagated said mitigating genetic trait is less fit than native algae or cyanobacteria not expressing said mitigating genetic trait.

According to yet another aspect of the present invention there is provided a method of obtaining a cultivated algae or cyanobacteria capable of mitigating the effects of self propagation or of introgression of at least one genetically engineered, commercially desirable genetic trait to an undesirable, uncultivated interbreeding species related to the cultivated algae or cyanobacteria, the method comprising transforming a population of the cultivated algae or cyanobacteria to co-express at least one genetically engineered, commercially desirable genetic trait, and at least one genetically linked, mitigating genetic trait, wherein said mitigating genetic trait is selected such that a self propagated or undesirable, species introgressing genes from the transgenic alga or cyanobacterium related to the cultivated algae or cyanobacteria expressing said mitigating genetic trait is less fit than an undesirable uncultivated interbreeding/introgressing species related to the cultivated algae or cyanobacteria not expressing said mitigating genetic trait, thereby obtaining a cultivated algae or cyanobacteria capable of mitigating the effects of introgression of the at least one genetically engineered, commercially desirable genetic trait of the cultivated algae or cyanobacteria to the undesirable, uncultivated interbreeding species related thereto.

According to further features in preferred embodiments of the invention described below, at least one commercially desirable genetic trait is selected from the group consisting of herbicide resistance, disease or zooplankton resistance, environmental stress resistance, the ability to fluoresce near ultraviolet light to photosynthetically usable light, high productivity, modified polysaccharide, protein or lipid qualities and quantities, enhanced yield, expression of heterologous products and other genetically modified algae and cyanobacteria products.

According to yet further features in preferred embodiments of the invention described below, the at least one mitigating genetic trait is selected from the group consisting of decreased RUBISCO, decreased storage or cell wall polysaccharides, decreased chlorophyll and/or carotene, decreased or eliminated motility organs, and increased non self metabolizable storage materials.

According to still further features in preferred embodiments of the invention described below, the at least one mitigating genetic trait is a reduced expression of endogenous genetic trait of said cultivated algae or cyanobacteria.

According to further features in preferred embodiments of the invention described below, the cultivated algae or cyanobacteria is one of the following Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp., Isochrysis sp. CS-177 Tetraselmis chuii CS-26 Tetraselmis suecica CS-187, and the commercially desirable genetic trait is single double or triple herbicide resistance, and the mitigating genetic trait is reduced RUBISCO, which is also commercially desirable.

According to further features in preferred embodiments of the invention described below, the cultivated algae or cyanobacteria is one of the following Synechococcus PCC7002, Phaeodactylum tricornutum Nannochlropiis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp, Isochrysis sp. CS-177 Tetraselmis chuii CS-26 Tetraselmis suecica CS-187, and the commercially desirable genetic trait is virus resistance, and the mitigating genetic trait is lack of motility organs, which is also commercially desirable.

According to further features in preferred embodiments of the invention described below, the cultivated algae or cyanobacteria is one of the following Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp, Isochrysis sp. CS-177, Tetraselmis chuii CS-26 Tetraselmis suecica CS-187, and the commercially desirable genetic trait is the ability to fluoresce near ultraviolet light as blue or red light, and the mitigating genetic trait is reduced photosystem II antennae size, which is also commercially desirable.

According to further features in preferred embodiments of the invention described below the cultivated algae or cyanobacteria is one of the following Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp, Isochrysis sp. CS-177 Tetraselmis chuii CS-26 Tetraselmis suecica CS-187, and the commercially desirable genetic trait is a protein content modified to high lysine and methionine, and the mitigating genetic trait is lack of reduced carotene synthesis as part of resistance to phytoene desaturase inhibiting herbicides.

According to further features in preferred embodiments of the invention described below, the cultivated algae or cyanobacteria is one of the following Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp. Isochrysis sp. CS-177 Tetraselmis chuii CS-26 Tetraselmis suecica CS-187, and the commercially desirable genetic trait is enhanced non self metabolizable storage polysaccharides, and the mitigating genetic trait is reduced storage starch formation.

According to another aspect of the present invention there is provided a genetic construct for mitigating the effects of establishment by self propagation or introgression of a genetically engineered commercially desirable genetic trait of a cultivated algae or cyanobacteria to an undesirable species related to the cultivated algae or cyanobacteria, the genetic construct comprising a first polynucleotide encoding the at least one commercially desirable genetic trait and a second polynucleotide encoding at least one mitigating genetic trait, wherein said at least one mitigating genetic trait is selected such that an undesirable, species related to the cultivated algae or cyanobacteria expressing said at least one mitigating genetic trait is less fit than the undesirable species related to the cultivated algae or cyanobacteria not expressing said at least one mitigating genetic trait and wherein expression of said commercially desirable and said at least one mitigating genetic trait is genetically linked, and a genetically modified cultivated algae or cyanobacteria comprising the genetic construct.

According to further features in preferred embodiments of the invention described below, said first and said second polynucleotides are covalently linked.

According to still further features in preferred embodiments of the invention described below, the first and said second polynucleotides are functionally linked.

According to yet further features in preferred embodiments of the invention described below, the first and second polynucleotides are co-transformed.

According to still further features in preferred embodiments of the invention described below, the first and second polynucleotides are integrated into the same chromosomal locus.

According to still further features in preferred embodiments of the invention described below, the first and second polynucleotides are integrated separately into an organism that has no known ability to exchange DNA among cells.

According to further features in preferred embodiments of the invention described below, the at least one commercially desirable genetic trait is selected from the group consisting of herbicide resistance, disease or zooplankton resistance, environmental stress resistance, the ability to fluorescence near ultraviolet light to photosynthetically usable light, high productivity, modified polysaccharide, protein or lipid qualities and quantities, enhanced yield, and expression of heterologous products and other genetically modified algae and cyanobacteria products.

The present invention successfully addresses the shortcomings of the presently known configurations by conceiving and providing a mechanism for mitigating the establishment of the transgenic algae or cyanobacteria and its progeny from establishing by self-propagation or the effects of introgression of a genetically engineered genetic trait of a alga or cyanobacteria to competing organisms. In the case of asexual organisms, where conjugation is unknown, it is sufficient that the mitigating gene be an irreversible mutation to a mitigating form.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1. Schematic diagram used to induce glyphosate resistant EPSPS in tandem with tla1 RNAi in C. reinhardtii. The coding region of EPSPS is cloned under the control of the RbcS promoter. The RNAi cassette of tla1 is cloned in tandem to the EPSPS, upstream to the RbcS terminator.

FIG. 2. Schematic diagram used to induce HPPD in background mutant algae with reduced antenna size. The coding region of HPPD is cloned under the control of the RbcS promoter.

FIG. 3. Schematic diagram used to induce the virus CAPSID and RbcS RNAi cassette in C. reinhardtii. The coding region of the CAPSID is cloned under the control of the RbcS promoter. The RbcS RNAi cassette is cloned in tandem to the CAPSID, upstream to the RbcS terminator.

FIG. 4. Schematic diagram used to induce the virus CAPSID and RbcS antisense in the green alga C. reinhardtii. The coding region of the CAPSID is cloned under the control of the RbcS promoter and upstream to the RbcS terminator. The RbcS is cloned in an antisense orientation in tandem to the CAPSID, downstream to the RbcS promoter and upstream to the RbcS terminator.

FIG. 5. Schematic diagram used to induce the virus CAPSID and RbcS antisense in cyanobacteria Synechococcus PCC7002. The coding region of the CAPSID is cloned under the control of the RbcS promoter and upstream to the RbcS terminator. The RbcS is cloned in an antisense orientation in tandem to the CAPSID, downstream to the RbcS promoter and upstream to the RbcS terminator.

FIG. 6. Schematic diagram used to induce PDS together with the high lysine BHL8 protein or high methionine 2S albumin protein in C. reinhardtii. The coding region of PDS is cloned under the control of the hsp70A and the RbcS promoters and upstream to the RbcS terminator. The BHL8 or 2S are cloned in tandem to PDS, downstream to the RbcS promoter and upstream to the RbcS terminator.

FIG. 7. Schematic diagram used to induce PDS together with the high lysine BHL8 protein or high methionine 2S albumin protein in cyanobacteria Synechococcus PCC7002. The coding region of PDS is cloned under the control of RbcS promoter and upstream to the RbcS terminator. The BHL8 of 2S are cloned in tandem to PDS, downstream to the RbcS promoter and upstream to the RbcS terminator.

FIG. 8. Schematic diagram used to induce the merA and merB genes together with RNAi of sta1 (A) and the 1-SST and 1-FFT genes (B) in C. reinhardtii. The coding region of merA is cloned under the control of the RbcS promoter and upstream to the RbcS terminator in tandem to merB, which is cloned under the control of the RbcS promoter and upstream to the RbcS terminator. This is in tandem to RNAi cassette of sta1 or sta6, which are cloned under the control of the RbcS promoter and upstream to the RbcS terminator. This together with the 1-SST and 1-FFT genes cloned each under the control of the RbcS promoter and upstream to the RbcS terminator.

FIG. 9. Schematic diagram used to induce the BFP in the C. reinhardtii mutant oda12-1. The coding region of BFP is cloned under the control of the RbcS promoter and upstream to the RbcS terminator.

FIG. 10. Schematic diagram used to induce the BFP and PilT antisense in cyanobacteria Synechococcus PCC7002. The coding region of BFP is cloned under the control of the RbcS promoter and upstream to the RbcS terminator. The PilT is cloned in an antisense orientation in tandem to BFP, downstream to the RbcS promoter and upstream to the RbcS terminator.

FIG. 11. Schematic diagram used to induce the PPO in the C. reinhardtii mutant oda 12-1.

### DETAILED DESCRIPTION OF THE INVENTION

The present invention is of genetic mechanisms that can be used for preventing the establishment of transgenic algae or cyanobacteria in natural ecosystems and mitigating the effects of introgression of a genetically engineered genetic trait of a cultivated algae or cyanobacteria to an undesirable, related species of the algae or cyanobacteria. Specifically, the present invention can be used to preclude the establishment of self-propagated transgenic algae or cyanobacteria and mitigating the effects of introgression of genetically engineered traits related algae or cyanobacteria.

The principles and operation of the present invention may be better understood with reference to the accompanying descriptions and examples.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Generally, the nomenclature used herein and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturers' specifications. Generally, the nomenclature used herein and the laboratory procedures utilized in the present invention include molecular, biochemical, microbiological and recombinant DNA techniques. Such techniques are thoroughly explained in the literature. See, for example, Sambrook et al., (1989); Ausubel, R. M., ed. (1994); Ausubel et al (1989); Perbal, (1988); Watson et al., (1998); methodologies as set forth in U.S. Pat. Nos. 4,666,828; 4,683,202; 4,801,531; 5,192,659 and 5,272,057; Cellis, J. E., ed. (1994); Coligan J. E., ed. (1994); Stites et al. (eds), (1994); Mishell and Shiigi (eds), (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; Gait, M. J., ed. (1984); Hames, B. D., and Higgins S. J., eds. (1985); Hames, B. D., and Higgins S. J., eds. (1984); “Freshney, R. I., ed. (1986);” (1986); Perbal, B., (1984) and “Methods in Enzymology” Vol. 1-317, Academic Press;” (1990); Marshak et al., (1996). Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

As used herein the term genetically linked refers to a genetic distance smaller than 50 centiMorgan, preferably smaller than 40 centiMorgan, more preferably smaller than 30 centiMorgan, more preferably smaller than 20 centiMorgan, more preferably smaller than 10 centiMorgan, more preferably smaller than 5 centiMorgan, more preferably smaller than 1 centiMorgan, most preferably in the range of 0 to 1 centiMorgan, wherein 0 centiMorgan refers to juxtaposed sequences.

One of the greatest advantages of herbicide-resistant algae and cyanobacteria is that they allow control of closely-related algae and cyanobacteria that have the same herbicide selectivity spectrum as the cultivated algae and cyanobacteria and could not be previously controlled. Similarly, an advantage of disease resistant algae and cyanobacteria is that they will not be decimated by pathogens. Highly productive algae and cyanobacteria are also advantageous, as are algae and cyanobacteria with modified product such as different types of starch and oils. These, and other genetic traits have been or could be transgenically introduced into algae or cyanobacteria of various types (see Table 1, herein below).

TABLE 1
Commercially desirable traits that can be engineered into
algae and cyanobacteria that may be undesirable if algae
or cyanobacteria released into natural ecosystems

| Trait | Genetic Element | Source |
|---|---|---|
| Fatty acid | delta(12)-fatty acid | various |
| composition | dehydrogenase (fad2), | |
| Fatty acid | fatty acid desaturase | various |
| composition | | |
| Fatty acid | thioesterase (TE) | Umbellularia californica |
| composition | | |
| Enhanced | Tla1 antennae. Similar | (see text) |
| photo- | acting genes, or antenna | |
| synthesis | mutant (advantageous in | |
| | high light only) | |
| Enhanced | Aldolase and TPI | (see text) |
| photo- | | |
| synthesis | | |
| Enhanced | Blue Fluorescent protein | (see text) |
| photo- | | |
| synthesis | | |
| Increased | Overexpressed cystathione γ- | Heterologous plant or |
| methionine | synthase | bacterial |
| content | | |
| Increased | Elevated dihydropicolinate | Mutant bacteria |
| lysine | synthase and | Endogenous antisense |
| content | suppressed lysine | or RNAi |
| | ketobutyrate reductase/ | |
| | saccharopine dehydrogenase | |
| Herbicide | 5-enolpyruvylshikimate-3- | Agrobacterium tumefaciens |
| resistance | phosphate synthase (EPSPS) | CP4 or Zea mays mutants |
| Herbicide | glyphosate oxidoreductase | Ochrobactrum anthropi |
| resistance | | |
| Herbicide | acetolactate synthase | Various sources |
| resistance | | |
| Herbicide | Nitrilase | Klebsiella pneumoniae |
| resistance | | subspecies ozanae |
| Herbicide | phosphinothricin N- | S. hygroscopicus or |
| resistance | acetyltransferase | S. viridochromogenes |
| Herbicide | 4-hydroxyphenyl-pyruvate- | Arabidopsis |
| resistance | dioxygenase (HPPD) | |
| Herbicide | Protoporphyrinogen oxidase | Amaranthus tuberculantus |
| resistance | (PPO) | |
| Mercury | merA + merB | Mercury resistant |
| volatili- | | bacteria |
| zation | | |
| Virus/phage | Helicase | From pathogens |
| resistance | | |
| Virus/phage | replicase | From pathogens |
| resistance | | |
| Virus | viral coat protein | From pathogens |
| resistance | | |

The advantages of transgenics are well appreciated, if there is no danger of establishment of the transgenic algae or cyanobacteria in natural ecosystems or introgression into a related alga or cyanobacterium. Because the advantages of transgenics are so great, as in the above cases, new modified transgenic algae and cyanobacteria are being developed.

Hence, while conceiving the present invention, the concept of mitigating the risks of establishment in natural ecosystems or introgression of a genetically engineered trait from the cultivated algae or cyanobacteria, it was conceived that the primary gene of choice having the desired trait should be in tandem constructs with “anti-establishment”, mitigating genes (Table 2) conferring a disadvantage on the algae or cyanobacteria when in natural ecosystems or into introgessed progeny, while being benign or advantageous to the cultivated algae or cyanobacteria. This coupling can either be physical, where the two genes are covalently linked prior to transformation or by the same physical juxtaposition commonly achieved by co-transformation. Both will heretofore be termed “tandem”, as the result in tightly linked genes. These would render individuals released to natural ecosystems unfit to act as competitors with its own wild type as well as other algae and cyanobacteria species.

In a special case, if the cultivated algae or cyanobacteria are asexual and non-conjugating, it is only necessary to mitigating the effects of self propagation. In that case the genetically engineered, commercially desirable genetic trait can be transformed into a population of the cultivated algae or cyanobacteria that express an irreversible (e.g. deletion) mutation conferring a mitigating trait. Such mutations exist in culture collections or can be obtained by mutagenesis, preferably by ultraviolet or gamma irradiation that causes deletions that cannot be reversed. Chemical mutagenesis, which typically causes point mutations in a single nucleotide can be reversed. Such mutations have been reported, e.g. in chloroplast antenna (Melis et al. 1998, Lee et al., 2002), with reduced RUBISCO (Khrebtukova and Spreitzer. 1996), lacking organs of motility (Okamoto and Ohmori 2002; Tanner et. al 2008). If the genetically engineered, commercially desirable genetic trait(s) is/are transformed into algae or cyanobacteria bearing such a natural or induced mutation that acts as a mitigating genetic trait, then they will be less fit than native algae or cyanobacteria and not be able to establish themselves in natural ecosystems.

As further detailed and exemplified herein below, genes that decrease RUBISCO or starch, remove cilia or other movement organelles among others would all be useful for that purpose, as they would often be benign or advantageous to the cultivated algae or cyanobacteria while detrimental establishment in the wild.

TABLE 2
Example of commercially desirable traits that can be engineered into
algae and cyanobacteria that would render algae or cyanobacteria unfit
and non-competitive if released into natural ecosystems.

| Trait | Genetic Element | Source |
|---|---|---|
| Lowered | Antisense or RNAi of large | Native |
| RUBISCO | and/or small RUBISCO subunit | |
| Reduced | tla1, or similarly acting | RNAi or antisense |
| photosystem | transgene, or a reduced antenna [symbol] | endogenous gene, |
| 2 | mutant | or mutagenesis |
| antenna | deleterious in low light in | |
| | natural ecosystems | |
| Reduced | Resistance to phytoene RbcS | Hydrilla |
| carotene | terminator desaturase inhibiting | resistant mutant |
| | herbicides | |
| Decreased | Sta-1 | RNAi or antisense |
| starch | | endogenous gene |
| Increased | 1-SST, 1FFT | RNAi or antisense |
| inulin | | endogenous gene |
| Modified | oda1-12, PilT | Chlamydomonas/ |
| flagella or [symbol] | | Synechococcus sp. |
| cilia | | PCC 7002 |
| Decreased | polysaccharide synthase | RNAi or antisense |
| cell wall | | endogenous gene |

[symbol]

indicates data missing or illegible when filed

A transgene encoding reduced content of RUBISCO (ribulose 1,5 bis phosphate carboxylase/oxygenase) (such as an antisense or RNAi construct of the small or the large subunit of RUBISCO) allows normal algae or cyanobacteria growth only at artificially high carbon dioxide concentrations.

A transgene such as tla1 or a mutation encoding reduced photosystem 2 antennae size, allows growth only at high light intensities but allows greater cell packing in commercial production facilities with less light energy wasted as heat; such organisms would not have enough chlorophyll to compete with indigenous organisms in natural ecosystems.

An antisense or RNAi construct of any of the genes encoding cilia or flagella (or similar motility organ) formation or action prevents the transgenic alga or cyanobacterium to position itself optimally based on environmental stimuli. Such movement is required to compete in natural ecosystems, but is unnecessary and wastes energy in commercial cultivation.

A transgenic mutant form of the phytoene desaturase (pds) gene conferring resistance to phytoene desaturase inhibiting herbicides synthesizes less beta-carotene. The herbicide resistance allows controlling unwanted species in commercial culture and the less carotene is of little consequence in dense commercial culture but provides photoprotection to organisms in the natural environments and organisms with less beta carotene are less competitive in the natural ecosystems.

A transgene in the anti-sense or RNAi form encoding one of the genes encoding one or more of the polymers of the cell walls causes the alga or cyanobacterium to form a thinner cell wall. This thinner cell wall is of little consequence in commercial production, and the cell walls are the least commercially valued part of the cell, but organisms with thinner cell walls cannot compete in the variable vicissitudes of environmental conditions in natural ecosystems.

A transgene encoding a storage polymer such as inulin, levan, or graminan that is not degradable for use as energy when needed, especially if coupled with RNAi or antisense form of for example starch phosphorylase encoding gene whereby energy storage as starch becomes prevented. This is desirable in commercial production when the new polymer has a greater value than starch, but renders an organism that cannot mobilize reserves, less fit in a natural environment where it cannot compete with organisms that can mobilize reserves in times of need.

Any other transgene that is neutral or beneficial to the algae or cyanobacteria when cultivated commercially, but renders the algae or cyanobacteria unfit to compete in natural ecosystems, overcoming any benefit that may derive form the transgene tandemly bound to it may be used as well.

The present invention also provides a genetic construct for mitigating the effects of establishment or introgression of a genetically engineered commercially desirable genetic trait of a cultivated alga or cyanobacterium. The genetic construct comprises a first polynucleotide encoding at least one commercially desirable genetic trait and a second polynucleotide encoding at least one mitigating genetic trait. Expression of the commercially desirable and the mitigating genetic trait is genetically linked. The polynucleotide encoding the first, primary genetic trait is preferably flanked on both sides by polynucleotides encoding the second, mitigating genetic trait, to thereby reduce the risk of losing the second, mitigating genetic trait due to mutation, etc.

However, it will be appreciated that in many cases while using conventional transformation techniques genetic traits carried on two different vectors integrate to the same locus.

Thus, according to a further aspect of the present invention there is provided a cultivated algae or cyanobacteria genetically modified to include the above described genetic constructs and to express the traits encoded thereby.

In one embodiment, the second, mitigating genetic trait is selected from the group consisting of reduced RUBISCO, reduced photosynthetic antennae, or reduced starch content or removal of cilia or other propelling organelles Numerous specific examples of such genetic traits are listed herein and are further discussed in the Examples section that follows.

One such mitigating trait is reduced RUBISCO content. Genetically reduced RUBISCO would be neutral or advantageous to the algae or cyanobacteria growing in saturating carbon dioxide, but deleterious to the algae or cyanobacteria in the wild, by themselves or in introgressed progeny, where carbon dioxide is limiting and there would not be enough enzyme to fix carbon dioxide.

Another such mitigating trait is decreased starch content. Such algae or cyanobacteria would be desirable as they would funnel more photosynthate to more valuable products, but without starch, such algae and cyanobacteria would not have the desired storage components to compete and exist in natural ecosystems

Yet another such mitigating trait is the reducing of the photosystem 2 antennae size by reducing the chlorophyll content (anti-sense or RNAi of the tla1 or similar transgene, or a mutation encoding a reduced antenna) or by reducing the carotenoid content (using the mutant pds gene conferring herbicide resistance to fluridone and related herbicides). This is advantageous in high light intensity photobioreactors and shallow ponds, as it allows more biomass production and less photoinhibition, but in light-limiting natural ecosystems is highly deleterious.

Still another such mitigating trait is using mutants that are obtained transgenically that are less mobile.

Thus, these anti-establishment in natural ecosystem, introgression-mitigating traits are combined, according to the present invention, with the desirable genetically engineered traits, which genetically engineered traits include, but are not limited to, traits imposing resistance to herbicides, disease, zooplankton pests, and pathogens, resistance to environmental stress such as, but not limited to, heat, salinity, etc., and traits affecting yield, modified product and by-product quality, bioremediation, as well as expression of heterologous products and genetically modified products such as starches and oils, etc. Such traits for which genes has been isolated are well known in the art, for example, genes modifying fatty content [delta(12)-fatty acid dehydrogenase (fad2), fatty acid desaturase, and thioesterase (TE)], PAT), herbicide tolerance genes that collaterally control many bacteria and fungal pathogens (5-enolpyruvylshikimate-3-phosphate synthase (EPSPS), acetolactate synthase, glyphosate oxidoreductase, nitrilase, phosphinothricin N-acetyltransferase), genes conferring favorable mutations (acetolactate synthase (ALS) and acetyl-CoA-carboxylase), and numerous viral resistance genes (helicase replicase and various specific viral coat protein genes). Additional suitable genes are listed in Table land summarized in many recent texts.

Once a gene responsible for a mitigating trait has been selected, it must be engineered for algal or cyanobacterial expression along with the desirable trait that confers an advantage thereto. To introduce such genes into algae or cyanobacteria, a suitable chimeric gene and transformation vector must be constructed. A typical chimeric gene for transformation will include a promoter region, a heterologous structural DNA coding sequences and a 3′ non-translated polyadenylation site for algae. A heterologous structural DNA coding sequence means a structural coding sequence that is not native to the algae or cyanobacteria being transformed. Heterologous with respect to the promoter means that the coding sequence does not exist in nature in the same gene with the promoter to that it is now attached. Chimeric means a novel non-naturally occurring gene that is comprised of parts of different genes. In preparing the transformation vector, the various DNA fragments may be manipulated as necessary to create the desired vector. This includes using linkers or adaptors as necessary to form suitable restriction sites or to eliminate unwanted restriction sites or other like manipulations that are known to those of ordinary skill in the art.

Promoters that are known or found to cause transcription of a selected gene or genes in plant and bacterial cells can be used to implement the present invention in algae or cyanobacteria, respectively. Such promoters may be obtained from plants, plant pathogenic bacteria or plant viruses and include, but are not necessarily limited to, strong constitutive promoter such as a 35S promoter (Odell et al (1985) Nature 313, 810-812), a 35S′3 promoter (Hull and Howell (1987) Virology 86, 482-493) and the 19S promoter of cauliflower mosaic virus (CaMV35S and CaMV19S), the full-length transcript promoter from the figwort mosaic virus (FMV35S) and promoters isolated from plant genes such as EPSP synthase, ssRUBISCO genes. Selective expression in green tissue can be achieved by using, for example, the promoter of the gene encoding the small subunit of Rubisco (European patent application 87400544.0 published Oct. 21, 1987, as EP 0 242 246). All of these promoters have been used to create various types of DNA constructs that have been expressed in plants. See, for example PCT publication WO 84/02913 (Rogers et al., Monsanto,). The particular promoter selected should be capable of causing sufficient expression to result in the production of an effective amount of the respective proteins to confer the traits.

A particularly useful promoter for use in some embodiments of the present invention is the full-length transcript promoter from the figwort mosaic virus (FMV35S). The FMV35S promoter is particularly useful because of its ability to cause uniform and high levels of expression in plant tissues. The DNA sequence of a FMV35S promoter is presented in U.S. Pat. No. 5,512,466 and is identified as SEQ ID NO:17 therein. The promoters used for expressing the genes according to the present invention may be further modified if desired to alter their expression characteristics. For example, the CaMV35S promoter may be ligated to the portion of the ssRUBISCO gene which represses the expression of ssRUBISCO in the absence of light, to create a promoter which is active in leaves but not in roots. The resulting chimeric promoter may be used as described herein. As used herein, the phrase “CaMV35S” or “FMV35S” promoter includes variations of these promoters, e.g., promoters derived by means of ligation with operator regions, random or controlled mutagenesis, addition or duplication of enhancer sequences, etc.

The 3′ non-translated region contains a polyadenylation signal that functions in algae (but not cyanobacteria) to cause the addition of polyadenylated nucleotides to the 3′ end of an RNA sequence. Examples of suitable 3′ regions are the 3′ transcribed, non-translated regions containing the polyadenylation signal of plant genes like the 7s soybean storage protein genes and the pea E9 small subunit of the RuBP carboxylase gene (ssRUBISCO).

The RNAs produced by a DNA construct of the present invention also preferably contains a 5′ non-translated leader sequence. This sequence can be derived from the promoters selected to express the genes, and can be specifically modified so as to increase translation of the mRNAs. The 5′ non-translated regions can also be obtained from viral RNA's, from suitable eukaryotic genes, or from a synthetic gene sequence. The present invention is not limited to constructs wherein the non-translated region is derived from the 5′ non-translated sequence that accompanies the promoter sequence. Rather, the non-translated leader sequences can be part of the 5′ end of the non-translated region of the native coding sequence for the heterologous coding sequence, or part of the promoter sequence, or can be derived from an unrelated promoter or coding sequence as discussed above.

In a preferred embodiment according to the present invention, the vector that is used to introduce the encoded proteins into the host cells of the algae or cyanobacteria will comprise an appropriate selectable marker. In a more preferred embodiment according to the present invention the vector is an expression vector comprising both a selectable marker and an origin of replication. In another most preferred embodiment according to the present invention the vector will be a shuttle vector, which can propagate both in E. coli (wherein the construct comprises an appropriate selectable marker and origin of replication) and be compatible for propagation or integration in the genome of the plant organism of choice. In yet another embodiment, the construct comprising the promoter of choice, and the gene of interest is placed in a viral vector which is used to infect the cells. This virus may be integrated in the genome of the organism of choice or may remain non-integrated.

According to some embodiments of the present invention secretion of the protein or proteins out of the cell is preferred. In this embodiment the construct will comprise a signal sequence to effect secretion as is known in the art. For some applications, a signal sequence that is recognized in the active growth phase will be most preferred. As will be recognized by the skilled artisan, the appropriate signal sequence should be placed immediately downstream of the translational start site (ATG), and in frame with the coding sequence of the gene to be expressed.

Introduction of the construct into the cells is accomplished by any conventional method for transfection, infection or the like as is known in the art including electroporation and biolistic transformations. In constructs comprising a selectable marker the cells may be selected for those bearing functional copies of the construct. If the plasmid comprising the gene of interest is episomal, the appropriate selective conditions will be used during growth. Stable transfectants and stable cell lines may be derived from the transfected cells in appropriate cases, in order to conveniently maintain the genotype of interest. Cell growth is accomplished in accordance with the cell type, using any standard growth conditions as may be suitable to support the growth of the specific cell line.

A DNA construct of the present invention can be inserted into the genome of algae or cyanobacteria by any suitable method. Such methods may involve, for example, the use of liposomes, electroporation, chemicals that increase free DNA uptake such as polyethylene glycol (PEG), vacuum filtration, particle gun technology (biolistic bombardment with tungsten or gold particles; see, for example, Sanford et al., U.S. Pat. No. 4,945,050; McCabe et al. (1988) Biotechnology, 6:923-926). Also see, Weissinger et al. (1988) Annual Rev. Genet., 22:421-477; Datta et al. (1990) Biotechnology, 8:736-740; Klein et al. (1988) Proc. Natl. Acad. Sci. USA, 85:4305-4309; Klein et al. (1988) Biotechnology, 6:559-563 (maize); Klein et al. (1988) Plant Physiol., 91:440-444; Fromm et al. (1990) Biotechnology, 8:833-839; and Tomes et al. “Direct DNA transfer into intact plant cells via microprojectile bombardment.” In: Gamborg and Phillips (Eds.) Plant Cell, Tissue and Organ Culture: Fundamental Methods; Springer-Verlag, Berlin (1995); Hooydaas-Van Slogteren & Hooykaas (1984) Nature (London), 311:763-764; Bytebier et al. (1987) Proc. Natl. Acad. Sci. USA, 84:5345-5349;) and other mechanical DNA transfer techniques, and transformation using viruses Such techniques include, but are not limited to, injection methods or microprojectile methods, as described in detail herein below. Application of these systems to different species depends upon the ability to regenerate that particular algal or cyanobacterial species from protoplasts.

An additional advantage of using a dual (tandem) system including a gene that may have an advantage in natural ecosystems with a mitigating gene is that the pair can be chosen in such a manner that one of the pair can have traits that will allow it to be used as a selectable marker, obviating the need for a separate selectable marker.

Confirmation of the transgenic nature of the algal or cyanobacterial cells may be performed by PCR analysis, antibiotic or herbicide resistance, enzymatic analysis and/or Southern blots to verify transformation. Progeny of the initial algal or cyanobacterial strains may be obtained by continuous sub-culturing may be obtained and analyzed to verify whether the transgenes are heritable. Heritability of the transgene is further confirmation of the stable transformation of the transgene in the algae or cyanobacteria. The transgenic algae or cyanobacteria are then grown and harvested using conventional procedures.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

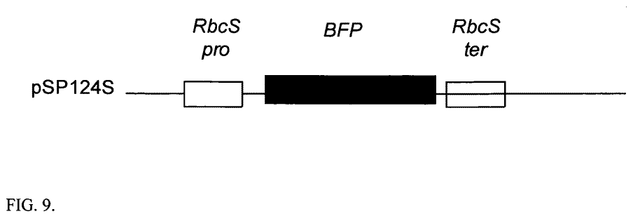

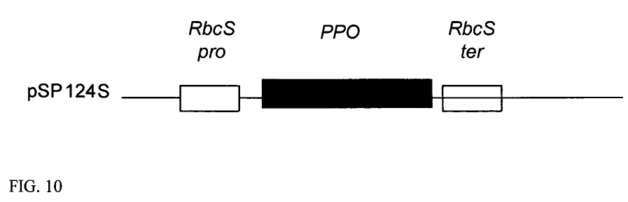

Reference is now made to the following examples, which together with the above descriptions, illustrate the invention in a non-limiting fashion. The concept of using genetic engineering to mitigate any positive effects transgenes may confer when released from controlled culture conditions into the natural environment, preventing the establishment of the transgenic algae or cyanobacteria and the products they may have from introgression to other species, is based on the following premise: If a transgene construct has in totality a small fitness disadvantage, it will remain localized as a very small proportion of the population. Therefore, gene establishment and flow should be mitigated by lowering the fitness of recipients below the fitness of the wild type so that they will not spread. This concept of “transgenic mitigation” (TM) was proposed for higher plants WO 04/46362, from which the present invention claims priority) and in a subsequent publication (Gressel, J. 1999: Tandem constructs: preventing the rise of superweeds. Trends Biotech. 17: 361-366, see FIGS. 9 and 10) in which mitigator genes are added to the desired primary transgene, which would reduce the fitness advantage to hybrids and their rare progeny, and thus considerably reduce risk. It is now extended to transgenic algae and cyanobacteria.

The TM approach is based on the facts that: 1) tandem constructs act as tightly linked genes, and their segregation from each other is exceedingly rare, far below the natural mutation rate; and 2) The TM traits chosen are selected to be nearly neutral or favorable to the cultivated crops, but deleterious to non-crop progeny (weeds, etc) due to a negative selection pressure; and 3) Individuals bearing even mildly harmful TM traits will be kept at exceedingly low frequencies in weed populations because weeds typically have a very high seed output and strongly compete amongst themselves, eliminating even marginally unfit individuals (Gressel, 1999). That this approach has been effective in higher plants has been illustrated in the following scientific publications: Al-Ahmad, et al., (2004; 2005; 2006, Al-Ahmad and Gressel, 2006).

Basically, in the cases of algae and cyanobacteria, these findings can be extended by tandemly combining almost any commercially useful trait that might spread in natural environments (Table 1) with a commercially neutral or advantageous trait that would render organisms unfit to compete in natural ecosystems (Table 2) as demonstrated in these few non-exclusive examples.

### Example 1

### Prevention of Establishment and Introgression of Glyphosate Herbicide Resistance by Coupling with Mutation in the tla1 Gene Conferring a Smaller Photosynthetic Antenna

One of the traits suitable for Transgenic Mitigation in constructs with a primary, desirable trait is down regulation of a form of the tla1 gene (such as GenBank Accession #AF534571) that reduces the number of chlorophyll molecules in the antennae of photosystem 2. Such strains can live only in the high light intensity of bioreactors and shallow ponds, where they allow greater packing, but cannot compete with the superior light capture of organisms with full size antennae. Such organisms with full size antennae are kept out of the culture ponds by having traits such as glyphosate herbicide resistance. Rare algae or cyanobacteria introgressing the TM construct could also no longer compete with native organisms in natural ecosystems. In order to determine whether co-transformation of a desirable transgene with a mitigator gene would prevent proliferation of transgenic strains having the tandem construct can compete in the case of breach of containment, a tandem construct was made containing an EPSP synthase gene (enolphosphate shikimate phosphate synthase) gene (SEQ ID NO: 1) for glyphosate herbicide resistance as the primary desirable gene, and a RNAi cassette of the tla1 gene (SEQ ID NO: 2) as a mitigator, and used to transform Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp., Isochrysis sp. CS-177 Tetraselmis chuii CS-26 Tetraselmis suecica CS-187.

### Experimental Procedures

Assembling the Tandem Construct

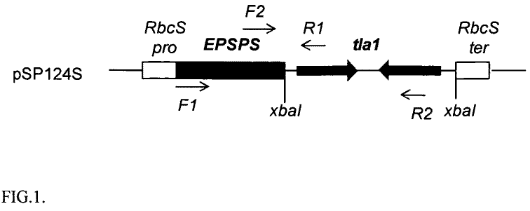

A DNA fragment corresponding to nucleotides 1 to 293 of the C. reinhardtii tla1 gene (GenBank accession #AF534571) is de novo synthesized in sense and antisense orientation with a 50-bp DNA spacer separating the sense and antisense fragments (SEQ ID NO:2). This fragment is cloned under the control of the C. reinhardtii rbcS2B promoter and downstream to the de novo synthesized EPSPS gene (SEQ ID NO:1) in the plasmid pSP124s (Lumbreras et al. 1998) to generate plasmid pEPSPS-tla1 (FIG. 1).

Another option: The de novo synthesized EPSPS gene for algae under the control of rbcS2 promoter and 3′rbcS2 terminator, downstream to the ble selectable marker in the plasmid pSP124s (Lumbreras, Stevens et al. 1998) is transformed into the C. reinhardtii tla1 mutant (Polle et al., 2003). The transformed algae will express the EPSPS gene on the background of the tla1 deficient mutant.

### Transformation of Algae

Algae cells in 0.4 ml of growth medium containing 5% PEG6000 were transformed with the pEPSPS-tla1 plasmid (1±5 mg) by the glass bead vortexing method (Kindle, 1990). The transformation mixture was then transferred to 10 ml of non-selective growth medium for recovery. The cells were kept for at least 18 h at 25° C. in the light. Cells were collected by centrifugation and plated at a density of 10⁸ cells per 80 mm plate. Transformants were selected on fresh TAP agar plates containing 10 mM glyphosate, for 7-10 days at 30° C. Conditions are modified for each organism according to its needs, based on modifications of standard protocols.

### Gene Integration Analyses of Algal or Cyanobacterial Transformants

Genomic DNA was isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA was isolated using either QIAGENS's Plant RNeasy Kit or the Trizol Reagent (Invitrogen, Carlsbad, Calif.).

The DNA was analyzed by PCR for the presence of in tact tandemly linked epsps and tla1 genomic insert. Four different DNA segments within the genomic TM T-DNA insert were amplified over the positions indicated in FIG. 1 with the following primers:

EPSPS forward primer 1 (SEQ ID NO:3):
TCCCCGGCGACAAGAGCAT
Tla1 reverse primer 1 (SEQ ID NO:4):
AAGAGCGCGGTTTGGTCAGC
EPSPS forward primer 2 (SEQ ID NO:5):
CACCGCATCGCCATGAGCTT
Tla1 Reverse primer 2 (SEQ ID NO:6):
GCTGACCAAACCGCGCTCTT

PCR reactions were carried out in 50 μL aliquots containing about 200 ng genomic DNA, 5 μL of 10×DyNAzyme™ II buffer (Finnzymes Oy, ESPOO, Finland), 1.5 U of DyNAzyme™ II DNA polymerase (Finnzymes Oy, ESPOO, Finland), 5 μL of 2.5 mM of each dNTP(s) (Roche Diagnostics, GmbH), and 35 pmol of each primer, in sterile distilled water. The mixture was denatured for 3 min at 94° C. and amplified for 35 cycles (94° C. for 30 s, 51° C. (DNA segments A and C) or 57° C. (segments B and D) for 30 s, 72° C. for 1 min) with a final cycle of 7 min at 72° C. The PCR products (15 μL) were loaded directly onto 1% (w/v) agarose gels to verify single bands. The remaining PCR products were purified using the QIAquick PCR Purification Kite (Qiagen, Hilden, Germany) according to the manufacturer's instructions, and sequenced to confirm the integration of the TM T-DNA.

In vivo epsps assay. Putative transformed algal or cyanobacterial cells were cultured in a solution of 10 mM glyphosate in standard algae or cyanobacteria culture media. At this concentration, all non-transgenic cells are killed.

In vivo tla1 assay Putatively transformed algae or cyanobacteria cells were diluted and plated out and cultured on agar plates in standard algae or cyanobacteria culture media such that single cells develop into colonies. These colonies were light yellow green in color vs. wild type colonies that are dark green in color.

Inheritance of the TM Construct Transgenes

Productivity of algae or cyanobacteria transformants at high light intensities: The transformants are analyzed for high productivity in full sunlight and lack of photoinhibition as previously demonstrated by United States Patent Application 20080120749 and by Poll et al., 2003.

Competition of TM transgenics with the wild type algae and cyanobacteria The transgenic TM algae or cyanobacteria were used to compete with natural species in simulated conditions. 1000 transgenic cells per ml were pipetted into unfiltered sea water in aquaria and cultivated in 100 μEin per cm² per sec light fluence, to simulate light conditions in the sea at a nominal depth. Aliquots were removed initially at daily, and later at weekly intervals, and the dwindling proportion of yellow green colonies are counted. Aliquots at the same dilutions were plated in parallel on the same media, but containing 10 mM glyphosate.

Within a few months, neither light yellow green colonies tla1 colonies nor glyphosate resistant colonies could be found in the aquaria. All glyphosate resistant colonies were light green in color, demonstrating that the tandem traits do not segregate.

### Example 2

### Prevention of Establishment and Introgression of HPPD Inhibiting Herbicide Resistance by Coupling with a Selected Mutation a Gene Conferring a Smaller Photosynthetic Antennae

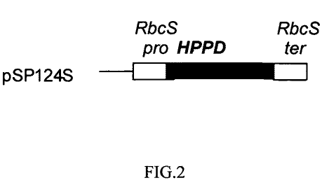

One of the traits suitable for Mitigation in constructs with a primary, desirable trait is down regulation of a form of the tla1 gene, as described in Example 1. Such modification of antenna size can be achieved by mutagenesis of the organism prior to introducing the commercial gene of choice. Such mutant strains can live only in the high light intensity of bioreactors and shallow ponds, where they allow greater packing, but cannot compete with the superior light capture of organisms with full size antennae. Such organisms with full size antennae are kept out of the culture ponds by having traits such as herbicide resistance, in this example resistance to inhibitors of the enzyme HPPD (4-hydroxyphenyl-pyruvate-dioxygenase). Rare escapes of algae or cyanobacteria bearing the HPPD gene in this background could no longer compete with native organisms in natural ecosystems. In order to determine whether transformation of a desirable transgene into an organism with a mutant mitigator gene would prevent proliferation of transgenic strains having the commercially desirable transgenic trait can compete in the case of breach of containment, a construct of FIG. 2 was made containing a HPPD (4-hydroxyphenyl-pyruvate-dioxygenase) (Accession no. AF000228) gene (SEQ ID NO:9) conferring resistance to the herbicide isoxaflutole (and related HPPD inhibiting herbicides), as the primary desirable gene, and used to transform Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp, Isochrysis sp. CS-177 Tetraselmis chuii CS-26 Tetraselmis suecica CS-187, bearing a mutation conferring small antennae size.

### Experimental Procedures

Attaining Reduced Antennae Mutants by UV Mutagenesis with Metronidazole Selection.

Metronidazole (2-methyl-5-nitroimidazole-1-ethanol) was shown to be effective for the selective enrichment of mutants of Chlamydomonas reinhardtii that possess impaired photosynthetic electron transport. More than 99.9% of wild-type cells were killed when incubated in the presence of 6-10 mM metronidazole for 24 hr under illumination of 7500 lux. Survival of wild-type cells in darkness and of mutants that are blocked at different steps in photosynthetic electron transport was nearly 100% when incubated in the presence of the drug under identical conditions (Schmidt et al. 1977).

We applied similar principles on Nannochloropsis sp. (strains CS 246 and CS179). Cells were grown in Artificial Sea Water (ASW) (Guillard, 1962) enriched with f/2. 25 ml of each cell culture in liquid media was placed in Petri dish and was exposed to UV irradiation (UV-C Lamp 30W) for 6.5 min. resulting in cell death of approximately 90%. The remaining cells were allowed a recovery time of 15 hrs. under dark conditions, then centrifuged (4000 rpm, 5 min.), re-suspended in 2 ml. 50-100 μl were plated on 1% Bacto Agar ASW plates containing 10 mM metronidazole+f/2 mineral supplement+10 mM HCO₃⁻. Light intensity was maintained at ˜2000 lux with light duration of 14 hrs followed by 10 hrs. dark. The temperature was kept at 25° C.

To assess physiological properties of reduced antennae sized algae compared with their relevant wild type strains we performed a set of procedures that enabled us to evaluate each strain.

Initially, each modified strain is checked for the trait modified, (reduced antennae size). A screening process is established where colonies of mutant algae are allowed to grow on metronidazole containing agar plates to verify that the desired trait, i.e. reduced antennae size has been established and is maintained. Next, pale green growing colonies where picked and transferred to liquid medium for further physiological evaluation.

This includes:
1. Growth rate
2. Photosynthetic activity
3. Respiration activity
4. Chlorophyll content

An overall report is generated for each strain that is used to estimate the feasibility of using the strain. Those with a near normal photosynthetic rate at high light intensities without photoinhibition, and a near normal growth rate are then used a mitigation platform for further development. That they are unable to compete with wild type algae under open sea conditions is ascertained as in example 1.

Assembling the HPPD Construct

The de novo synthesized HPPD gene for algae is cloned under the control of rbcS2 promoter and 3 rbcS2 terminator, downstream to the ble selectable marker in the plasmid pSP124s (Lumbreras et al. 1998) and transformed into the algae bearing a mutation conferring small antennae size.

### Transformation of Algae

Algae cells in 0.4 ml of growth medium containing 5% PEG6000 were transformed with the pHPPD plasmid (1±5 mg) by the glass bead vortexing method (Kindle, 1990). The transformation mixture was then transferred to 10 ml of non-selective growth medium for recovery. The cells were kept for at least 18 h at 25° C. in the light. Cells were collected by centrifugation and plated at a density of 10⁸ cells per 80 mm plate. Transformants were selected on fresh TAP agar plates containing 10 mM isoxaflutole, for 7-10 days at 30° C. Conditions are modified for each organism according to its needs, based on modifications of standard protocols.

### Gene Integration Analyses of Algal or Cyanobacterial Transformants

Genomic DNA was isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA was isolated using either QIAGENS's Plant RNeasy Kit or the Trizol Reagent (Invitrogen, Carlsbad, Calif.).

The DNA was analyzed by PCR for the presence of intact HPPD insert as indicated in FIG. 2 with the following primers:

HPPD forward primer 1 (SEQ ID NO:7):
ATGG GCCACCAAAA CGCCGC
HPPD reverse primer 2 (SEQ ID NO:8):
CCCACTAACTGTTTGGCTTC

PCR reactions were carried out in 50 μL aliquots containing about 200 ng genomic DNA, 5 μL of 10×DyNAzyme™ II buffer (Finnzymes Oy, ESPOO, Finland), 1.5 U of DyNAzyme™ II DNA polymerase (Finnzymes Oy, ESPOO, Finland), 5 μL of 2.5 mM of each dNTP(s) (Roche Diagnostics, GmbH), and 35 pmol of each primer, in sterile distilled water. The mixture was denatured for 3 min at 94° C. and amplified for 35 cycles (94° C. for 30 s, 51° C. (DNA segments A and C) or 57° C. (segments B and D) for 30·s, 72° C. for 1 min) with a final cycle of 7 min at 72° C. The PCR products (15 μL) were loaded directly onto 1% (w/v) agarose gels to verify single bands. The remaining PCR products were purified using the QIAquick PCR Purification Kit® (Qiagen, Hilden, Germany) according to the manufacturer's instructions, and sequenced to confirm the integration of the TM T-DNA.

In vivo HPPD assay. Putative transformed algal or cyanobacterial cells were cultured in a solution of 10 mM isoxaflutole in standard algae or cyanobacteria culture media. At this concentration, all non-transgenic cells are killed.

Productivity of algae or cyanobacteria transformants at high light intensities: The transformants are analyzed for high productivity in full sunlight and lack of photoinhibition as previously demonstrated by United States Patent Application 20080120749 and by Poll et al., 2003.

Competition of TM transgenics with the wild type algae and cyanobacteria The transgenic TM algae or cyanobacteria were used to compete with natural species in simulated conditions. 1000 transgenic cells per ml were pipetted into unfiltered sea water in aquaria and cultivated in 100 μEin per cm² per sec light fluence, to simulate light conditions in the sea at a nominal depth. Aliquots were removed initially at daily, and later at weekly intervals, and the dwindling proportion of yellow green colonies are counted. Aliquots at the same dilutions were plated in parallel on the same media, but containing 10 mM isoxaflutole.

Within a few months, neither light yellow green colonies mutant colonies nor isoxaflutole resistant colonies could be found in the aquaria. All isoxaflutole resistant colonies were light green in color, demonstrating that the traits do not segregate.

### Example 3

### Prevention of Establishment and Introgression of Virus Resistance by Coupling with the Transgenes Conferring a Lowered RUBISCO Content

One of the traits suitable for Transgenic Mitigation in constructs with a primary, desirable trait is using an antisense or RNAi form of one or both of the subunits of the RUBISCO gene (such as GenBank Accessions XM_{—}001702356, NC_{—}005353) that cause the reduction of the number of RUBISCO molecules in the algae or cyanobacteria. Such strains can live only in the carbon dioxide levels artificially created by using carbon dioxide enrichment—such as from flue gasses from industrial sources to facilitate high levels of carbon fixation in bioreactors and shallow ponds. In this situation, lower levels of the low affinity RUBISCO are needed as the carbon dioxide levels in the ponds are at least 100 fold greater than ambient levels. Algae and cyanobacteria with less RUBISCO cannot compete in natural ecosystems with the superior carbon dioxide capture of native organisms with full RUBISCO content at the low ambient levels of carbon dioxide. Such organisms with full RUBISCO complement are kept out of the culture ponds by having traits such as herbicide resistance (Example 1). Rare algae or cyanobacteria introgressing the TM construct can also no longer compete with native organisms in natural ecosystems. In order to determine whether co-transformation of a desirable transgene with a mitigator gene would prevent proliferation of transgenic strains having the tandem construct can compete in the case of breach of containment, a tandem construct is made containing a desirable gene encoding a gene conferring virus resistance (GenBankAccession #M85052) as the primary desirable gene, and the rubisco mutant gene as a mitigator (GenBank accession #XM_{—}001702356). Genes conferring resistance to viruses or phages specifically pathogenic to Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PC

### Assembling the Tandem Construct

Generation of Chlamydomonas reinhardtii Expressing the Virus CAPSID and the RNAi of rbcS2B Under the Control of the rbcS2 Promoter

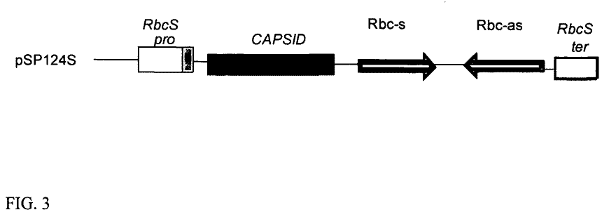

The chlorella virus capsid sequence is chemically synthesized using the published sequence (Accession M85052) with modifications according to the codon usage of the green algae Chlamydomonas reinhardtii (SEQ ID NO: 12). The gene is cloned under the control of the RbcS2 promoter in the plasmid pSP124S (Sizova et al. 2001). For generation of RNAi of rbcS2B, a 248 bp fragment corresponding to the coding sequence of rbcS is amplified with primers: TCTAGA CTGCAG CGCCGTCATTGCCAAGTCCT (SEQ ID NO: 10) adding XbaI and PstI and GGATCC AAGCTT AATGTAGTCGACCTGGGCGG (SEQ ID NO:11) adding BamHI and HindIII restriction in their 5′ flanking region. The PCR product is cloned in forward and reverse orientations into the PstI/BamHI and HindIII/XbaI sites of the pSTBlue-1 vector (Novagen, Madison, Wis., USA), flanking a 200-bp DNA spacer previously inserted into the EcoRV site. The rbcS RNAi cassette is then excised from pSTBlue-1 by XbaI digestion and is cloned downstream to the virus capsid in the corresponding site of pSP124S (Lumbreras, Stevens et al. 1998) (FIG. 3).

Generation of Chlamydomonas reinhardtii Expressing the Virus CAPSID and the Antisense rbcS2B Gene Under the Control of the rbcS2 Promoter

The chlorella virus CAPSID sequence is chemically synthesized using the published sequence (Accession M85052) with modifications according to the codon usage of the green algae Chlamydomonas reinhardtii (SEQ ID NO: 12). The gene is cloned into pGEM-T vector (Promega) and then transferred under the control of the RbcS2 promoter in the plasmid pSP124S (Sizova et al. 2001).

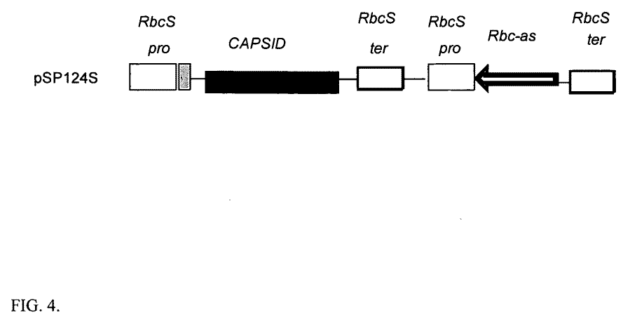

For generation of antisense of rbcS2B gene, the 567 bp fragment of the C. reinhardtii rbcS2B gene (SEQ ID NO:57) is PCR amplified with the forward primer: TCTAGA ATGGCCGCCGTCATTGCCAAG (SEQ ID NO: 13) and the reverse primer: TCTAGA ACGAGCGCCTCCATTTACACG (SEQ ID NO: 14), containing the XbaI site in their 5′ region, and is cloned into pGEM-T (Promega). The XbaI fragment is then cloned downstream to the virus capsid in the corresponding site of pSP124S (Lumbreras, Stevens et al. 1998) (FIG. 4).

### Transformation of Algae

Algae cells in 0.4 ml of growth medium containing 5% PEG6000 are transformed with the pCAPSID-(anti)RUBISCO plasmid (1±5 mg) by the glass bead vortexing method (Kindle, 1990). The transformation mixture is then transferred to 10 ml of non-selective growth medium for recovery. The cells are kept for at least 18 h at 25° C. in the light. Cells were collected by centrifugation and plated at a density of 10⁸ cells per 80 mm plate. Selection is made according to (Van Etten et al., 1983). Briefly, transformants are grown to a density of 2×10⁷ to 3×10⁷ algae per milliliter, concentrated by centrifugation, and resuspended in MBBIM (Van Etten et al., 1983) at 38×10⁷ algae per milliliter. Two hundred microliters of algae (7.6×10⁷ algae) plus 100 μl of appropriate dilutions of the virus are added to 2.5 ml of 0.7 percent agar in MBBM (48° C. to 50° C.) and immediately overlaid on petri plates containing 15 ml of MBBM plus 1.5 percent agar. The plates are then incubated at 25° C. in continuous light. Plaques are visible after 2-4 days. Conditions are modified for each organism according to its needs, based on modifications of standard protocols.

Generation of Cyanobacteria Synechococcus PCC7002 Expressing the Virus Capsid and the Antisense of rbcS Gene Under the Control of the rbcS2 Promoter

The Synechococcus virus (Syn9) capsid gene is chemically synthesized using the published sequence (Accession 4239190) (SEQ ID NO:15) and is directly cloned downstream to the RbcS promoter in the pCB4 plasmid.

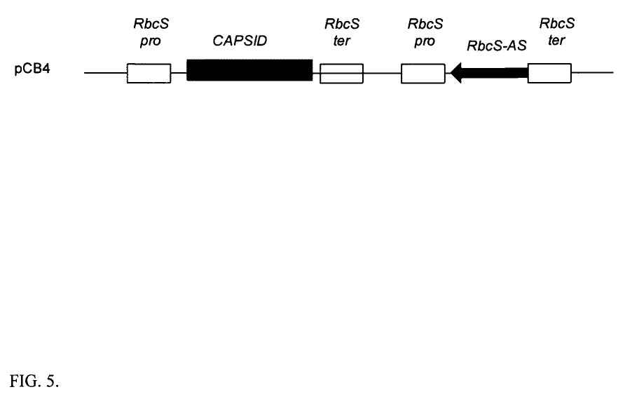

The coding sequence of Synechococcus PCC7002 rbcS2B (SEQ ID NO:66) is amplified using the forward primer: (SEQ ID NO:16) and the reverse primer: GTAACGGGTTTGGTTGGGC (SEQ ID NO: 17) harboring BamHI restriction sites in their 5′ ends, followed by cloning into pGEM-T plasmid (Promega). The 333 bp fragment is then excised from the pGEM-T plasmid and cloned into the BamHI site in the shuttle vector pCB4 in an antisense orientation downstream to the virus capsid gene (FIG. 5).

### Transformation of Cyanobacteria

For transformation of Synechococcus PCC7002, cells are cultured in 100 ml of BG-11 liquid medium at 28° C. under white fluorescent light and subcultured at the mid-exponential phase of growth. To 1.0 ml of cell suspension containing 2×10⁸ cells, which are cultured at the mid-exponential phase of growth, 0.5 or 1.0 μg of donor DNA (in 10 mM Tris/1 mM EDTA, pH 8.0) is added, and the mixture is incubated in the dark at 26° C. overnight. After incubation for a further 6 h in the light, the transformants are selected on BG-11 agar plates using the plaque selection assay which is preformed as described in (Wilson et al. 1993). Serial dilutions of the cyanophage filtrates are added to separate 0.5-ml volumes of a 40× concentration (ca. 8×10⁹ cells ml⁻¹) of exponentially growing Synechococcus PCC7002 that are incubated at 25° C. for 1 h with occasional agitation to encourage cyanophage adsorption. Each phage-cell suspension is then added to 2.5 ml of 0.4% molten ASW agar (42° C.); these suspensions are mixed gently and then poured evenly onto a solid 1% ASW agar plate (diameter, 85 mm) before being left to set at room temperature for 1 h. Incubation of the plates is carried out at 25° C. under constant illumination (15 to 25 microeinsteins m⁻² s⁻¹), and the plates are monitored daily for the formation of plaques. Conditions are modified for each organism according to its needs, based on modifications of standard protocols.

### Gene Integration Analyses of Algal or Cyanobacterial Transformants

Genomic DNA was isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA was isolated using either QIAGENS's Plant RNeasy Kit or the Trizol Reagent (Invitrogen, Carlsbad, Calif.).

The DNA was analyzed by PCR for the presence of intact tandemly linked PBCV-1 virus CAPSID-gene encoding resistance to Chlamydomonas virus, and low rubisco genomic insert. Four different DNA segments within the genomic TM T-DNA insert were amplified over the positions indicated in FIG. 4 with the following primers for algae:

For CAPSID and RNAi of RUBISCO:
CAPSID Forward primer:

| | |
|---|---|
| ATGGCCGGCGGCCTGAGCCA | (SEQ ID NO:18) |

Rubisco reverse of RNAi Reverse primer:

| | |
|---|---|
| AATGTAGTCGACCTGGGCGG | (SEQ ID NO:19) |

For capsid and antisense of rubisco:
CAPSID Forward primer:

| | |
|---|---|
| ATGGCCGGCGGCCTGAGCCA | (SEQ ID NO:20) |
| rubisco Reverse primer: | |
| GTGTAAATGGAGGCGCTCGT | (SEQ ID NO:21) |

And the following primers for cyanobacteria
CAPSID Forward primer:

| | |
|---|---|
| ATGTCTTTCCAAAACCTCCA | (SEQ ID NO:22) |
| rubisco Reverse primer: | |
| AGCCCAACCAAACCCGTTAC | (SEQ ID NO:23) |

In vivo virus assay. Putatively transformed and serially diluted algae or cyanobacteria cells are cultured in Petri dishes on standard algae or cyanobacteria culture media in an incubator with high carbon dioxide until colonies derived from single cells are apparent. The dishes are then inoculated with a solution containing virus, such that all non-transformed cells are killed.

In vivo rubisco assay Healthy colonies from the algae or cyanobacteria cells are picked and plated in marked replicates on agar plates in standard algae or cyanobacteria culture media. Two replicates of each colony are cultured in ambient carbon dioxide and two in >10% carbon dioxide. The colonies that grow the largest on the high carbon dioxide but hardly develop on ambient carbon dioxide are candidates for verification by western immunoblotting with commercial anti-RUBISCO antibody to demonstrate that indeed the amount of RUBISCO per cell is quite reduced

Competition of TM transgenics with the wild type algae and cyanobacteria The transgenic TM algae or cyanobacteria are used to compete with natural species in simulated conditions. 1000 transgenic cells per ml are pipetted into unfiltered sea water in aquaria and cultivated in 100 μEin per cm² per sec light fluence, to simulate light conditions in the sea at a nominal depth at ambient carbon dioxide levels. Aliquots are removed initially at daily, and later at weekly intervals, and the dwindling proportion of virus resistant colonies are counted.

### Results

Within a few months, no virus-resistant colonies can be found in the aquaria.

### Example 4

### Prevention of Establishment of Balanced Protein Containing Transgenic Lines of Algae by Engineering Balanced Protein into Algae with Fitness-Lowering Phytoene Desaturase Herbicide Resistance

One of the traits suitable for Transgenic Mitigation in constructs with a primary, desirable trait is using a mutant form of the pds (phytoene desaturase) gene (such as GenBank Accession AY639658) that confers resistance to the herbicide fluridone and related herbicides, but also reduces the carotene levels leaving algae more subject photoinhibition and to UV light induced damage. Such strains can live only in the high light intensity of bioreactors and shallow ponds, but in dense cultures, but cannot compete with the superior light capture of organisms with full size antennae with its complete complement of carotenoids. Such organisms with full size antennae are kept out of the culture ponds by having traits such as herbicide resistance, which is needed to prevent contamination in any event. Rare algae or cyanobacteria introgressing the TM construct could also no longer compete with native organisms in natural ecosystems. In order to determine whether co-transformation of a desirable transgene with a mitigator gene would prevent proliferation of transgenic strains having the tandem construct can compete in the case of breach of containment, a tandem construct was made. This tandem construct contained a modified high lysine or a modified high methionine or a fusion storage protein containing high levels of both as one of the primary desirable genes together with expression of feedback insensitive bacterial DHDPS (dihydrodipicolinate synthase) and with RNAi of LKR/SDH (lysine-ketoglutarate reductase/saccharopine dehydrogenase) as described previously (Zhu and Galili, 2004) to increase free lysine level or overexpression of cystathionine 7-synthase (CGS). CGS is the enzyme that controls the synthesis of the first intermediate metabolite in the methionine pathway to increase free methionine (Avraham et al., 2005). In addition the construct contained mutant pds gene (phytoene desaturase) gene (GenBank accession #AY639658) for herbicide resistance as a mitigator gene as well as a desirable gene in its own right, and as a selectable marker to isolate transform ants. The construct was used to transform Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp., Isochrysis sp. CS-177 Tetraselmis chuii CS-26 Tetraselmis suecica CS-187.

### Experimental Procedures

### Assembling the Tandem Construct

Generation of Chlamydomonas reinhardtii Expressing PDS Together with the High Lysine BHL8 Protein or High Methionine 2S Albumin Protein

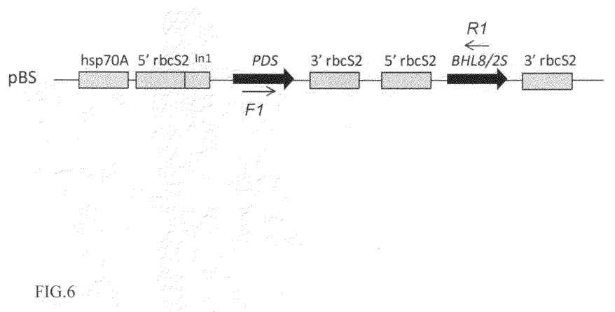

For expression the de novo synthesized pds gene (SEQ ID NO:24) together with the high lysine BHL8 protein coding gene (SEQ ID NO:25) or high methionine 2S albumin coding gene (SEQ ID NO:26) in C. reinhardtii, the two genes are cloned under the control of the C. reinhardtii rbcS2 promoter and terminator and then combined into pSP124s replacing the ble selectable marker coding sequence (FIG. 6).

Generation of Cyanobacteria Synechococcus PCC7002 Expressing PDS Together with the High Lysine BHL8 Protein or High Methionine 2S Albumin Protein

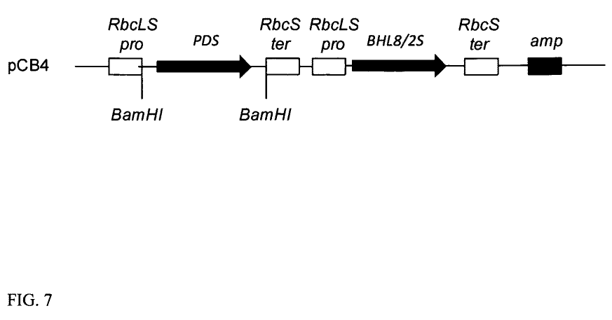

For cyanobacteria, the de novo synthesized pds (SEQ ID NO:27) genes together with high lysine BHL8 protein coding gene (SEQ ID NO:28) or high methionine 2S albumin coding gene (SEQ ID NO:29) are cloned under the control of the constitutive promoter of the rbcLS operon (Deng and Coleman 1999) in the plasmid pCB4 by BamHI restriction sites, as well into various expression vectors, allowing various levels of expression driven by different promoters, including constitutive, inducible, and log phase temporal promoters (FIG. 7).

Transformation into algae was conducted as described in example 1.

### Transformation of Cyanobacteria

For transformation of Synechococcus PCC7002, cells are cultured in 100 ml of BG-11 liquid medium at 28° C. under white fluorescent light and subcultured at the mid-exponential phase of growth. To 1.0 ml of cell suspension containing 2×10⁸ cells, which are cultured at the mid-exponential phase of growth, 0.5 or 1.0 μg of donor DNA (in 10 mM Tris/1 mM EDTA, pH 8.0) is added, and the mixture is incubated in the dark at 26° C. overnight. After incubation for a further 6 h in the light, the transformants are directly selected on BG-11 agar plates containing 1.5% agar, 1 mM sodium thiosulfate and fluridone. The transformation frequency is calculated by counting the number of transformants.

### Gene Integration Analyses of Algae or Cyanobacteria Transformants

Genomic DNA is isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA was isolated using either QIAGENS's Plant RNeasy Kit or the Trizol Reagent (Invitrogen, Carlsbad, Calif.).

The DNA is analyzed by PCR for the presence of intact tandemly linked pds and BHL8 genomic insert. Four different DNA segments within the genomic TM T-DNA insert were amplified over the positions indicated in FIGS. 6 and 7 with the following primers:

forward primer: (SEQ ID NO:30):
ATGACTGTTGCTAGGTCGGT (PDS)
Reverse primer: (SEQ ID NO:31):
GCACTTGGGGGTCTTGGCGA (BHL8)

PCR reactions is carried out in 50 μL aliquots containing about 200 ng genomic DNA, 5 μL of 10×DyNAzyme™ II buffer (Finnzymes Oy, ESPOO, Finland), 1.5 U of DyNAzyme™ II DNA polymerase (Finnzymes Oy, ESPOO, Finland), 5 μL of 2.5 mM of each dNTP(s) (Roche Diagnostics, GmbH), and 35 pmol of each primer, in sterile distilled water. The mixture is denatured for 3 min at 94° C. and amplified for 35 cycles (94° C. for 30 s, 51° C. (DNA segments A and C) or 57° C. (segments B and D) for 30 s, 72° C. for 1 min) with a final cycle of 7 min at 72° C. The PCR products (15 μL) are loaded directly onto 1% (w/v) agarose gels to verify single bands. The remaining PCR products are purified using the QIAquick PCR Purification Kit® (Qiagen, Hilden, Germany) according to the manufacturer's instructions, and sequenced to confirm the integration of the TM T-DNA.

In vivo pds assay. Putative algae or cyanobacteria cells are cultured in a solution of 100 μM fluridone in standard algae or cyanobacteria culture media and cultured in high light. At this concentration, all non-transgenic cells are bleached and eventually die.

In vivo meth/lys protein assay Picked colonies putative algae or cyanobacteria cells are separately cultured in wells of 96 well microplate dishes in standard algae or cyanobacteria culture media containing fluridone, until the cultures become dense. Dimethyl sulfoxide is added to each well and the cultures are allowed to bleach in the light. The bleached cultures are reacted with a fluorescent tagged antibody specific for the high lysine/methionine storage protein used in the transformation and the levels are measured with a microplate reader. Crude proteins are isolated from the cultures indicated to have acceptable growth rates and high levels antigenic proteins, digested and the proportion of lysine and methionine in crude protein is measured in a Moore-Stein type automated amino-acid analyzer.

Inheritance of the TM Construct Transgenes

Productivity of algae or cyanobacteria transformants at high light intensities: The transformants are analyzed for high productivity in full sunlight and freedom from undesirable algae and cyanobacteria in the presence of fluridone.

Competition of TM transgenics with the wild type algae and cyanobacteria The transgenic TM algae or cyanobacteria are used to compete with natural species in simulated conditions. 1000 transgenic cells per ml are pipetted into unfiltered sea water in aquaria and cultivated in 100 μEin per cm² per sec light fluence, to simulate light conditions in the sea at a nominal depth. Aliquots are removed initially at daily, and later at weekly intervals, and plated on dishes with and without fluridone. The dwindling proportion of fluridone resistant colonies are counted.

### Results

Within a few months, fluridone resistant colonies cannot be found in the aquaria.

### Example 5

### Prevention of Establishment of Algal Individuals Transgenically Resistant to PPO Inhibiting Herbicides by Engineering PPO Resistance into Mutant Algae Lacking Motility

One of the traits suitable for Transgenic Mitigation in constructs with a primary, desirable trait is to have cells incapable of phototactic/chemotactic or thermotactic motility such that they cannot swim in a direction that is optimal survival (such as oda1-12 mutant for Chlamydomonas and the PilT accession NC_{—}010475 for cyanobacteria). Such strains do not need motility to exist in continually mixed high cell density bioreactors and ponds, but cannot compete with native organisms in a natural ecosystem, where they must be able to swim towards optimal light and away from danger. Because they do not waste energy on motility, they have more energy available for making commercially needed components Potentially contaminating cells organisms are kept out of the culture ponds by having traits such as herbicide resistance. Rare non-motile algae or cyanobacteria introgressing the TM construct could also no longer compete with native organisms in natural ecosystems. In order to determine whether co-transformation of a desirable transgene with a mitigator gene prevents proliferation of transgenic strains having the tandem construct can compete in the case of breach of containment, a construct was made containing a PPO (protoporphyrinogen oxidase) gene (Accession no. DQ386114) (SEQ ID NO: 32) conferring pyrazoxyfen herbicide resistance (and other PPO related herbicides) as the primary desirable gene, in the background of the oda12-1 mutant in algae or in tandem to the PilT gene (SEQ ID NO:33) (GenBank accession #NC_{—}010475) from cyanobacteria conferring non swimming as a mitigator, and used to transform Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp., Isochrysis sp. CS-177 Tetraselmis chuii CS-26 Tetraselmis suecica CS-187. Transformants were selected on fresh TAP agar plates containing 10 mM pyrazoxyfen, for 7-10 days at 30° C. Conditions are modified for each organism according to its needs, based on modifications of standard protocols

### Experimental Procedures

### Assembling the Tandem Construct

Generation of Chlamydomonas Containing a PPO Herbicide Resistant Gene in the Background of the oda12-1 Mutant

The oda12-1 mutant lacks the entire LC2+LC10 genes. This strain exhibits a flagellar beat frequency that is consistently less than that observed for strains that fail to assemble the entire outer arm and docking complex (Tanner 2008). Therefore the PPO herbicide resistant construct is transformed in the background of the oda12-1 mutant.

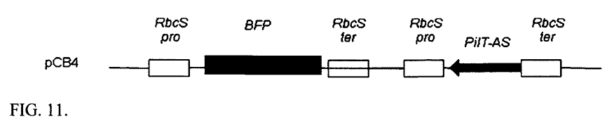

The PPO herbicide resistant sequence (Patzoldt 2006) is synthesized using the published sequence (Accession no. DQ386114) (SEQ ID NO:32) with modifications according to the codon usage of the green algae C. reinhardtii. The gene is cloned into pGEM-T vector (Promega) and then transferred into pSP124S (Sizova et al. 2001) under the control of the RbcS2 promoter and 3′ RbcS2 terminator. FIG. 11.

Another option is to produce either RNAi or antisense constructs directed against dynein heavy/light chains in tandem with the PPO herbicide resistant gene under the control of the RbcS2 promoter and 3′ RbcS2 terminator in the pSP124S vector.

### Transformation of Algae

Algae cells in 0.4 ml of growth medium containing 5% PEG6000 are transformed with plasmid from examples 1 and 2 (1±5 mg) by the glass bead vortexing method (Kindle 1990) or electroporation (Chow and Tung 1999). The transformation mixture is then transferred to 10 ml of non-selective growth medium for recovery. The cells are kept for at least 18 h at 25° C. in the light. Cells are collected by centrifugation and plated at a density of 10⁸ cells per 80 mm plate.

For selection transformants were grown on fresh TAP agar plates containing 10 mM pyrazoxyfen, for 7-10 days at 30° C. Conditions are modified for each organism according to its needs, based on modifications of standard protocols

Generation of Cyanobacteria Synechococcus PCC7002 Expressing a PPO Herbicide Resistant Gene and the Antisense of PilT Gene Under the Control of the rbcS2 Promoter

The PPO herbicide resistant gene (Patzoldt 2006) is chemically synthesized using the published sequence (Accession no: DQ386114) (SEQ ID NO:32) with modifications according to the codon usage of Synechococcus PCC7002 (SEQ ID NO:35) and with the addition of BamHI restriction sites in its both ends. The gene is cloned into pGEM-T vector (Promega) and then transferred into the BamHI site of pCB4 plasmid (Deng and Coleman, 1999) downstream to the Synechococcus rbcLS promoter and upstream to antisense of PilT gene (SEQ ID NO:33), followed by rbcLS terminator.

The coding sequence of Synechococcus PCC7002 PilT gene is amplified using the forward primer: ATGGATTACATGATCGAAGA (SEQ ID NO:36) and the reverse primer: GCGACGTTTTGCGGTTGGGC (SEQ ID NO:37) followed by cloning into pGEM-T plasmid (Promega). The fragment is then excised from the pGEM-T plasmid and cloned into the shuttle vector pCB4 in an antisense orientation downstream to the PPO herbicide resistant gene.

### Transformation of Cyanobacteria

For transformation of Synechococcus PCC7002, cells are cultured in 100 ml of ASN-III liquid medium at 28° C. under white fluorescent light and subcultured at the mid-exponential phase of growth. To 1.0 ml of cell suspension containing 2×10⁸ cells, which are cultured at the mid-exponential phase of growth, 0.5 or 1.0 μg of donor DNA (in 10 mM Tris/1 mM EDTA, pH 8.0) is added, and the mixture is incubated in the dark at 26° C. overnight. For selection transformants were grown on fresh TAP agar plates containing 10 mM pyrazoxyfen, for 7-10 days at 30° C. Conditions are modified for each organism according to its needs, based on modifications of standard protocols. Surviving cells are then transferred for future culturing and further examination.

### Gene Integration Analyses of Algae or Cyanobacteria Transformants

Genomic DNA is isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA is isolated using either QIAGENS's Plant RNeasy Kit or the Trizol Reagent (Invitrogen, Carlsbad, Calif.).
The DNA is analyzed by PCR for the presence of intact PPO. primers for algae:

| | |
|---|---|
| Forward primer: | |
| ATGGTAATTC AATCCATTAC | (SEQ ID NO:38) |
| Reverse primer: | |
| CGGTCTTCTCATCCATCTTC | (SEQ ID NO:39) |

And the following primers for cyanobacteria:

(SEQ ID NO:40)

Forward primer: CTGGACTCTC ATATATACGT from the PPO

(SEQ ID NO 41)

Reverse primer: CAACCGCAAAACGTCGCTAA from PilT

In vivo PPO assay. Putative transformed algal or cyanobacterial cells were cultured in a solution of 10 mM pyrazoxyfen in standard algae or cyanobacteria culture media. At this concentration, all non-transgenic cells are killed.

In vivo motility assay Picked pyrazoxyfen resistant algae or cyanobacteria cells are placed on a microscope slide and observed. The slide is then unilaterally illuminated and movement towards or away from the light (depending on intensity) is observed in wild type but not mutant cells.

Competition of TM transgenics with the wild type algae and cyanobacteria The transgenic TM algae or cyanobacteria are used to compete with natural species in simulated conditions. 1000 transgenic cells per ml are pipetted into unfiltered sea water in aquaria and cultivated in 100 μEin per cm² per sec light fluence, to simulate light conditions in the sea at a nominal depth. Aliquots are removed initially at daily, and later at weekly intervals and plated out, and the dwindling proportion of colonies that fluoresce in blue light are counted.

### Results

Within a few months, blue fluorescing colonies cannot be found in the aquaria.

### Example 6

### Prevention of Establishment of Algal Individuals Transgenically Resistant to Mercury in Flue Gas by Engineering them into Mutant Algae Lacking the Ability to Store Complex Carbohydrates

One of the traits suitable for Transgenic Mitigation in constructs with a primary, desirable trait is using an antisense or RNAi form of starch synthesizing genes that confer non-storage of starch (such as sta1, GenBank Accession: XM_{—}001693395) coupled to genes encoding enzymes responsible for the synthesis of polysaccharides such a inulin (such as 1-SST and 1-FFT, GenBank Accessions: AJ009757, AJ009756) or levan (such as SacB from Bacillus subtilis (NC-000964) or Bacillus amyloliquifaciens (NC_{—}009725) or levan sucrase gene from Erwinia amylovora (AJ831832) or ftf gene from Streptococcus mutans, GenBank Accession: NC_{—}004350) or graminan, a highly branched levan found in wheat, barley and other graminae that the algae or cyanobacteria are capable of storing but are incapable of mobilizing in times of need. Such strains can live only in bioreactors and ponds, where other traits are separately used to prevent the establishment of competing organisms. Rare algae or cyanobacteria introgressing the TM construct in natural ecosystems could also no longer compete with native organisms in the natural ecosystems. In order to determine whether co-transformation of a desirable transgene with a mitigator gene would prevent proliferation of transgenic strains having the tandem construct can compete in the case of breach of containment, the merA (SEQ ID NO:64; GenBank accession number NC_{—}002134) and merB genes (SEQ ID NO:65, GenBank accession number U77087) conferring mercury resistance are used as the primary desirable genes, and a tandem construct is made containing the above starch reducing/inulin over-producing genes as a mitigator and used to transform Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp., Isochrysis sp. CS-177 Tetraselmis chuii CS-26 Tetraselmis suecica CS-187. In this case the mercury resistant trait is used as a selectable marker.

### Experimental Procedures

### Assembly the Tandem Construct

Generation of Chlamydomonas reinhardtii Expressing RNAi of sta1 (for Reduced Starch) Together with the merA and merB Genes Conferring Mercury Resistance and the 1-SST and 1-FFT Genes Encoding Enzymes Responsible for the Synthesis of Inulin

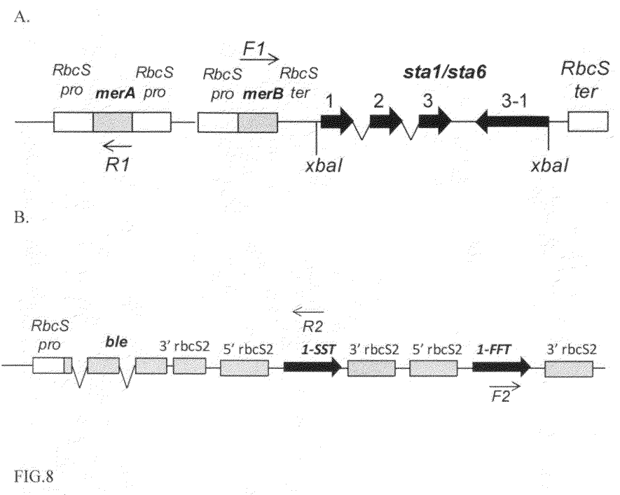

For generation of a tandem construct including the RNAi of C. reinhardtii sta1 gene encodes for AGPase large subunit (which are later used together with the merA and merB genes conferring mercury resistance), The primer for sta1 is: GCTCTAGAGCATGC TGTTAATGGCGACGCCTGG (SEQ ID NO: 42), and primer: GC GGATCCAAGCTT GAACCACTCCTTGTCGGTGG (SEQ ID NO:43) containing the XbaI+SphI and BamHI+HindIII restriction in their 5′ flanking region are used for amplification of exons number 2, 3, 4 and introns 2, 3 (597-1649 gDNA) of C. reinhardtii sta1 gene (SEQ ID NO: 67) using gDNA (genomic DNA) as a template and exons 2, 3, 4 (40-504 CDS) of C. reinhardtii sta1cDNA using cDNA as template. The 1053 bp genomic fragment was cloned into pSTBlue-1 (Novagene) in SphI/BamHI restriction sites and the 465 bp cDNA fragment is cloned into HindIII/XbaI sites of the same pSTBlue-1 plasmid in antisense orientation downstream to the sense genomic sequence. The sta1 RNAi cassette is then excised from pSTBlue-1 by XbaI digestion and cloned into the corresponding site of pSP124s, in the ble 3′UTR. Then a de novo synthesized merA and merB is synthesized according to the appropriate codon usage of the desired algae, each cloned under the control of rbcS2 promoter and terminator, is introduced into the same plasmid replacing the ble selectable marker, upstream to the sta1 RNAi cassette (FIG. 8A).

For generation of a construct including genes encoding enzymes responsible for the synthesis of inulin (such as Helianthus tuberosus 1-SST (SEQ ID NO: 44) and 1-FFT (SEQ ID NO:45); GenBank Accessions: AJ009757, AJ009756, respectively) or levans, such as SacB from Bacillus subtilis (NC 000964) (SEQ ID NO:69) or Bacillus amyloliquifaciens (NC_{—}009725) or levan sucrase gene from Erwinia amylovora (AJ831832) or fif gene from Streptococcus mutans, GenBank Accession: NC_{—}004350) the coding sequence of each gene is de novo synthesized according to the appropriate codon usage of the desired algae and cloned under the control of rbcS2 promoter and terminator. The tandem construct is introduced into the pSP124s plasmid downstream to the ble 3′UTR (FIG. 8B).

Co-transformation of the two plasmids into Chlamydomonas reinhardtii is analyzed using inverse PCR with specific primers (arrows indicates primers positions in FIGS. 8A and B).

Generation of Cyanobacteria Synechococcus PCC7002 Expressing Antisense of sta1 (for Reduced Starch) Together with the merA and merB Genes Conferring Mercury Resistance and the 1-SST and 1-FFT Genes Encoding Enzymes Responsible for the Synthesis of Inulin

For cyanobacteria the glgC (glucose-1-phosphate adenyltransferase) (SEQ ID NO:68) is cloned in antisense orientation using primers:

### Primer: GTGTGTTGTTGGCAATCGAG (SEQ ID NO:46) and

Primer: CTAGATTACCGTGCCGTCGG (SEQ ID NO:47) for amplification of glgC cDNA from Synecocysitis PCC 6803 and the PCR product is cloned into pGEM-T T/A vector (Promega). The fragment containing the complete AGPase coding sequence is cloned into the plasmid pCB4 in an antisense orientation downstream to the rbcLS promoter. Then the de novo synthesized merA and merB synthesized according to the cyanobacteria codon usage of the desired algae each cloned under the control of rbcLS promoter and terminator, is introduced into the same plasmid upstream to the glgC antisense sequence.

For generation of a construct including genes encoding enzymes responsible for the synthesis of inulin (such as Helianthus tuberosus 1-SST and J-FFT, GenBank Accessions: AJ009757, AJ009756) the coding sequence of each gene is de novo synthesized according to the appropriate codon usage of the desired cyanobacteria and cloned under the control of rbcLS promoter and terminator in pCB4 plasmid. Co-transformation of the two plasmids into Synechococcus PCC7002 is analyzed using inverse PCR with specific primers and genomic DNA as a template.

### Gene Integration Analyses of Algae or Cyanobacteria Transformants

Genomic DNA is isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA is isolated using either QIAGENS's Plant RNeasy Kit or the Trizol Reagent (Invitrogen, Carlsbad, Calif.).

The DNA is analyzed by inverse PCR as described by Ochman et al., 1998, for the presence of intact tandem linked merA, merB, and 1-FFT, 1-SST genomic inserts. Two different DNA segments within the genomic TM T-DNA insert are amplified over the positions indicated in FIG. 8 with the following primers:

Forward primer 1 (SEQ ID NO:48):
TCTCATCGCATTGCGCTGCA (merB)
Reverse primer 1 (SEQ ID NO:49):
CCAACTTTCCTGGAACCCGC (merA)
forward primer 2 (SEQ ID NO:50):
CACGTTTAGTTCCCATGATC (FFT-1)
Reverse primer 2 (SEQ ID NO:51):
CAAGCGTGGAACACATCTAC (SST-1)

The PCR products are purified using the QIAquick PCR Purification Kite (Qiagen, Hilden, Germany) according to the manufacturer's instructions, and sequenced to confirm the integration of the TM T-DNA.

In vivo merA/merB assay. Putative algae or cyanobacteria cells are plated on 5-10 μM PMA (phenyl mercury acetate, replacing methyl mercury) in standard algae or cyanobacteria culture media. At this concentration, all non-transgenic cells are killed.

In vivo starch assay Mercury resistant transgenic algae or cyanobacteria cells from numbered colonies of picked cells are cultured in 96-well dishes in standard algae or cyanobacteria culture media such that single cells develop into cultures. When cultures are dense, they are bleached with DMSO as in Example 3, and then stained for the absence of starch with iodine/potassium iodide solution.

Inulin determination The best cultures, most rapidly growing mercury resistant/low starch cultures able to attain the most dense growth are analyzed for inulin content according to Cairns (2003) and references therein.

Competition of TM transgenics with the wild type algae and cyanobacteria The transgenic TM algae or cyanobacteria are used to compete with natural species in simulated conditions. 1000 transgenic cells per ml are pipetted into unfiltered sea water in aquaria and cultivated in 100 μEin per cm² per sec light fluence, to simulate light conditions in the sea at a nominal depth. Aliquots are removed initially at daily, and later at weekly intervals, and the dwindling proportion of yellow green colonies are counted. Aliquots at the same dilutions were plated in parallel on the same media, but containing 10 μM PMA.

### Results

Within a few months, no methyl mercury resistant cells can be found in the aquaria.

### Example 7

### Prevention of Establishment and Introgression of Enhanced Photosynthesis and UV Resistance by Coupling with a Gene Preventing Flagela/Cilia Formation Resulting in Cells Lacking Self Motility

One of the traits suitable for Transgenic Mitigation in constructs with a primary, desirable trait is to have cells incapable of phototactic/chemotactic or thermotactic motility such that they cannot swim in a direction that is optimal survival (such as oda1-12 mutant for Chlamydomonas and the PilT accession NC_{—}010475 for cyanobacteria). Such strains do not need motility to exist in continually mixed high cell density bioreactors and ponds, but cannot compete with native organisms in a natural ecosystem, where they must be able to swim towards optimal light and away from danger. Because they do not waste energy on motility, they have more energy available for making commercially needed components Potentially contaminating cells organisms are kept out of the culture ponds by having traits such as herbicide resistance. Rare non-motile algae or cyanobacteria introgressing the TM construct could also no longer compete with native organisms in natural ecosystems. In order to determine whether co-transformation of a desirable transgene with a mitigator gene prevents proliferation of transgenic strains having the tandem construct can compete in the case of breach of containment, a tandem construct was made containing a BFP-blue fluorescing protein (SEQ ID NO:53) that converts cell damaging near ultraviolet light to blue light that can be used in photosynthesis, and the oda12-1 mutant in algae/PilT gene in cyanobacteria conferring non swimming as a mitigator (GenBank accession #NC 010475), and used to transform Synechococcus PCC7002, Phaeodactylum tricornutum, Nannochloropsis sp CS 246, Nannochloropsis oculata, Nannochloropsis salina, Pavlova lutheri CS182, Synechococcus PCC7942, Synechosystis PCC6803, Chlamydomonas reinhardtii, Chlorella vulgaris, Chlorella ssp., Isochrysis sp. CS-177 Tetraselmis chuii CS-26 Tetraselmis suecica CS-187. The resistance to UV damage and the ability to photosynthesize the blue fluorescence emanating from incident near UV light is used as a selectable marker for transformations.

### Experimental Procedures

### Assembling the Construct

Generation of Chlamydomonas Containing an BFP-Blue Fluorescing Protein Gene in the Background of oda12-1 Mutant

The oda12-1 mutant lacks the entire LC2+LC10 genes. This strain exhibits a flagellar beat frequency that is consistently less than that observed for strains that fail to assemble the entire outer arm and docking complex (Tanner 2008). Therefore the BFP-azurite construct is built in with the background of the oda12-1 mutant.

The BFP-azurite construct sequence (Mena et al. 2006) is chemically synthetized using the published sequence with modifications according to the codon usage of the green algae C. reinhardtii (SEQ ID NO:54).

The gene is cloned into pGEM-T vector (Promega) and then transferred into pSP124S (Sizova et al. 2001). under the control of the RbcS2 promoter and 3′ RbcS2 terminator (FIG. 9).

Another option is to produce either RNAi or antisense constructs directed against dynein heavy/light chains in tandem with the BFP-blue fluorescing protein gene under the control of the RbcS2 promoter and 3′ RbcS2 terminator in the pSP124S vector.

### Transformation of Algae

Algae cells in 0.4 ml of growth medium containing 5% PEG6000 are transformed with plasmid from examples 1 and 2 (1±5 mg) by the glass bead vortexing method (Kindle 1990) or electroporation (Chow and Tung 1999). The transformation mixture is then transferred to 10 ml of non-selective growth medium for recovery. The cells are kept for at least 18 h at 25° C. in the light. Cells are collected by centrifugation and plated at a density of 10⁸ cells per 80 mm plate. Transformants are grown on fresh TAP agar plates for 7 days in 30° C.

For selection, transformants are grown on fresh TAP agar plates for 7 days at 30° C. Colonies are transferred to micro-well plates at a dilution of 1-2 cells per microwell using medium, and cultured under UV light until it is apparent that there cells growing in most wells. BFP fluorescence is monitored at excitation of 383 nm and emission of 450 nm. DsRed and any other FP used are monitored with their specific excitation and emission spectra. Cells from microwells producing the highest fluorescent signal are collected and cultured as single cell colonies under UV light (duration and intensity are set at LD 99% of wild type cells). Surviving cells are then transferred for future culturing and further examination.

Generation of Cyanobacteria Synechococcus PCC7002 Expressing a BFP-Blue Fluorescing Protein Gene and the Antisense of PilT Gene Under the control of the rbcS2 Promoter

The BFP-azurite sequence (Mena et al. 2006) is chemically synthesized using the published sequence (SEQ ID NO:52) with modifications according to the codon usage of Synechococcus PCC7002 (SEQ ID NO:55) and with the addition of BamHI restriction sites in its both ends. The gene is cloned into pGEM-T vector (Promega) and then transferred into the BamHI site of pCB4 plasmid (Deng and Coleman, 1999) downstream to the Synechococcus rbcLS promoter and upstream to antisense of PilT gene (SEQ ID NO:53), followed by rbcLS terminator.

The coding sequence of Synechococcus PCC7002 PilT gene is amplified using the forward primer: ATGGATTACATGATCGAAGA (SEQ ID NO:58) and the reverse primer: GCGACGTTTTGCGGTTGGGC (SEQ ID NO:59) followed by cloning into pGEM-T plasmid (Promega). The fragment is then excised from the pGEM-T plasmid and cloned into the shuttle vector pCB4 in an antisense orientation downstream to the BFP gene (FIG. 11).

### Transformation of Cyanobacteria

For transformation of Synechococcus PCC7002, cells are cultured in 100 ml of ASN-III liquid medium at 28° C. under white fluorescent light and subcultured at the mid-exponential phase of growth. To 1.0 ml of cell suspension containing 2×10⁸ cells, which are cultured at the mid-exponential phase of growth, 0.5 or 1.0 μg of donor DNA (in 10 mM Tris/1 mM EDTA, pH 8.0) is added, and the mixture is incubated in the dark at 26° C. overnight. For selection, transformants are grown on fresh TAP Agar plates for 7 days at 30° C. Colonies are transferred to micro-well plates at a dilution of 1-2 cells per microwell using medium, and cultured with a 16/8 h light/dark period under white fluorescent light at 30° C. until it is apparent that there cells growing in most wells. BFP fluorescence is monitored at excitation of 383 nm and emission of 450 nm. DsRed and any other FP used are monitored with their specific excitation and emission spectra. Cells from microwells producing the highest fluorescent signal are collected and cultured as single cell colonies under UV light (duration and intensity are set at LD 99% of wild type cells). Surviving cells are then transferred for future culturing and further examination.

### Gene Integration Analyses of Algae or Cyanobacteria Transformants

Genomic DNA is isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA is isolated using either QIAGENS's Plant RNeasy Kit or the Trizol Reagent (Invitrogen, Carlsbad, Calif.).

The DNA is analyzed by PCR for the presence of intact tandemly linked BFP-blue fluorescing protein gene and PilT gene in an antisense orientation. Four different DNA segments within the genomic TM T-DNA insert are amplified over the positions indicated in FIG. 11 with the following primers for algae:

| | |
|---|---|
| Forward primer: | |
| ATGAGCAAGGGCGAGGAGCT | (SEQ ID NO:60) |
| Reverse primer: | |
| GTGGTGGTGGTGGTGGTGCT | (SEQ ID NO:61) |

And the following primers for cyanobacteria:

(SEQ ID NO:62)

Forward primer: AACACCACCACCACCACCAC from the BFP

(SEQ ID NO 63)

Reverse primer: CAACCGCAAAACGTCGCTAA from PilT

In vivo UV resistance assay. Putative algae or cyanobacteria cells are plated on Petri dishes and placed in a box where the sole irradiation is near UV light. Green colonies that develop are expected to contain the BFP gene.

In vivo motility assay Picked UV resistant algae or cyanobacteria cells are placed on a microscope slide and observed. The slide is then unilaterally illuminated and movement towards or away from the light (depending on intensity) is observed in wild type but not mutant cells.

Competition of TM transgenics with the wild type algae and cyanobacteria The transgenic TM algae or cyanobacteria are used to compete with natural species in simulated conditions. 1000 transgenic cells per ml are pipetted into unfiltered sea water in aquaria and cultivated in 100 μEin per cm² per sec light fluence, to simulate light conditions in the sea at a nominal depth. Aliquots are removed initially at daily, and later at weekly intervals and plated out, and the dwindling proportion of colonies that fluoresce in blue light are counted.

### Results

Within a few months, blue fluorescing colonies cannot be found in the aquaria.

### REFERENCES

Al-Ahmad H., and Gressel J. (2006) Mitigation using a tandem construct containing a selectively unfit gene precludes establishment of Brassica napus transgenes in hybrids and backcrosses with weedy Brassica rapa. Plant Biotechnology Journal 4:23-33.
Al-Ahmad, H. I., S. Galili and J. Gressel (2004) Tandem constructs to mitigate transgene persistence: tobacco as a model. Molecular Ecology 13:697-710
Al-Ahmad, H., S. Galili, and J. Gressel (2005) Poor competitive fitness of transgenically mitigated tobacco in competition with the wild type in a replacement series. Planta 222:372-385
Al-Ahmad H., Dwyer J., Moloney M., and Gressel J. (2006) Mitigation of establishment of Brassica napus transgenes in volunteers using a tandem construct containing a selectively unfit gene. Plant Biotechnology Journal 4:7-21
Al-Kaff, N. S., S, N. Covey, M. M. Kreike, A. M. Page, R. Pinder and P. J. Dale., 1998. Transcriptional and postranslational plant gene silencing in response to a pathogen. Science 279:2113-2115.
Anon., 1994a. Assessment criteria for determining environmental safety of plants with novel traits. Directive Dir. 94-08 Plant Products Division, Agriculture and Agri-Food Canada, Nepean Ontario. (http://www.cfia-acia.agr.ca/English/food/pbo/dir9408. html).
Anon., 1994b. The biology of Brassica napus L (canola/rapeseed). Directive Dir. 94-09 Plant Products Division, Agriculture and Agri-Food Canada, Nepean Ontario. (http://www.cfia-acia.agr.ca/English/food/pbo/dir9409.html).
Anon., 1997 Consensus document on the biology of Brassica napus L. (oilseed rape). Series on the harmonization of regulatory oversight in biotechnology No. 7. Environ. Directorate. Org. for Econ. Co-op. and Devel., Paris.
Arias, D. M. and L. H. Riesenberg., 1994. Gene flow between cultivated and wild sunflowers. Theor. Appl. Genet. 89:655-660.
Aukerman, M. J., M. Hirschfeld, L. Wester, M. Weaver, T. Clack, R. M. Amasino and R. A. Sharrock., 1997. A deletion in the PHYD gene of the Arabidopsis Wassilewskija ecotype defines a role for phytochrome D in red/far-red light sensing. Plant Cell 9:1317-1326.
Avraham T, Badani H, Galili S, Amir R (2005) Enhanced levels of methionine and cysteine in transgenic alfalfa (Medicago sativa L.) plants over-expressing the Arabidopsis cystathionine gamma-synthase gene. Plant Biotechnol J 3: 71-79 Azpiroz, R., Y. Wu, J. C. LoCascio and K. A. Feldmann., 1998. An Arabidopsis brassinosteroid-dependent mutant is blocked in cell elongation. Plant Cell 10: 219-230.
Baker, H. G., 1974. The evolution of weeds. Ann. Rev. Ecol. Sys. 5:1-24.
Baker, H. G., 1991. The continuing evolution of weeds. Econ. Bot. 45:445-449.
Barrett, S. C. H., 1983. Crop mimicry in weeds. Econ. Bot. 37:255-282.
Be, D. B., S. G. Rogers, T. B. Stone, and F. S. Serdy., 1996. Herbicide tolerant plants developed through biotechnology: Regulatory considerations in the United States. p. 341-346. In: S. O. Duke (ed.), Herbicide resistant crops: Agricultural, environmental, economic, regulatory, and technical aspects. CRC Press, Boca Raton.
Bergelson, J., C. B. Purrington, C. J. Palm, and J. C. Lopez-Guitierrez., 1996. Costs of resistance—a test using transgenic Arabidopsis thaliana. Proc. Roy. Soc. Lond. B. Biol. Sci. 262: 1659-1663.
Bewley, J. D., 1997. Breaking down the walls—a role for endo-beta-mannanase in release from seed dormancy? Trends Plant Sci. 2: 464-469.
Bing, D. B., R. K. Downey, and G. F. W. Rakow., 1996 Assessment of transgene escape from Brassica rapa (B. campestris) into B. nigra or Sinapis arvensis. Plant Breed. 115:1-4.
Boudry, P., M. Mirchen, P. Saumitou-Laprade, H, Vernet, and H. Van Dijk., 1993. The origin and evolution of weed beets: consequences for the breeding and release of herbicide-resistant transgenic sugar beets. Theor. Appl. Genet. 87:471-478.
Brown, J., and A. P. Brown., 1996. Gene transfer between canola (Brassica napus L. and B. campestris L.) and related weed species. Ann. Appl. Biol. 129:513-522.
Cairns A J (2003) Fructan biosynthesis in transgenic plants. J Exp Bot 54: 549-567
Choe, S. B. P. Dilkes, S. Fugioka, S. Takatsuto, A. Sukarai, and K. A. Felmann., 1998. The DWF4 gene of Arabidopsis encodes a cytochrome P450 that mediates multiple 22α-hydroxylation steps in brassinosteroid biosynthesis. Plant Cell 10:231-144.
Conner, A. J., and P. J. Dale., 1996. Reconsideration of pollen dispersal data from field trials of transgenic potatoes. Theor. Appl. Genet. 92:505-508.
Cooper, J. I. and A. F. Raybould., 1997. Transgenes for stress tolerance: consequences for weed evolution. Brighton Crop Protect. Conf.-Weeds. pp. 265-272.
Crawford, J., G. Squire, and D. Burn., 1997. Modeling spread of herbicide resistant oilseed rape. In: A. J. Gray, C. Glidden and F. Amjee (eds.). Environmental Impact of Genetically Modified Crops. Dept. of Environment, London, (in press).
Crawley, M. J., R. S. Hails, M. Rees, D. Kohn, and J. Buxton., 1993. Ecology of transgenic oilseed rape in natural habitats. Nature 363:620-623.
Darmency, H., 1994. The impact of hybrids between genetically modified crop plants and their related species: introgression and weediness. Mol. Ecol. 3:37-40.
DeKathen, A., 1998. The debate on risks from plant biotechnology: the end of reductionism? Plant Tissue Culture and Biotechnology 4: 136-148.
Deng M D, Coleman J R (1999) Ethanol synthesis by genetic engineering in cyanobacteria. Appl Environ Microbiol 65: 523-528
Devlin, P. F., S. R. Patel and G. C. Whitelam., 1998. Phytochrome E influences internode elongation and flowering time in Arabidopsis. Plant Cell 10:1479-1488.
Diepenbrock, W. and J. Leon., 1988. Quantitative effects of volunteer plants on glucosinolate content in double-low rapeseed (Brassica napus L.): a theoretical approach. Agronomie 8:373-377.
Eijlander, R., and W. J. Stiekema., 1994. Biological containment of potato (Solanum tuberosum): outcrossing to the related wild species black nightshade (Solanum nigrum) and bittersweet (Solanum dulcamara). Sex Plant Reprod. 7: 29-40.
Foley, M. E., and S. A. Fennemore., 1998. Genetic basis for seed dormancy. Seed Sci. Res. 8:173-182.
Galun, E. and A. Breiman., 1997. Transgenic Plants. Imperial College Press, London, 376 pp.
Goldburg, R., J. Rissler, H, Shand, and C. Hassebrook., 1990. Biotechnology's bitter harvest. Environmental Defense Fund, New-York.
Gould, F., 1991. The evolutionary potential of crop pests. Amer. Sci. 79: 496-507.
Gressel, J., 1997. Genetic engineering can either exacerbate or alleviate herbicide resistance. Proc. 50th New Zealand Plant Protection Conf. p. 298-306.
Gressel, J. 1999: Tandem constructs: preventing the rise of superweeds. Trends Biotech. 17: 361-366.
Gressel, J., 2008a Genetic Glass Ceilings, Transgenics for Crop Biodiversity, Johns Hopkins University Press, Baltimore.
Gressel, J. (2008) Transgenics are imperative for biofuel crops. Plant Science 174: 246-263.
Gressel, J., and A. W. Rotteveel, 2000. Risks from Biotechnologically-Derived Herbicide-Resistant Crops: Decision Trees for Assessment. Plant Breeding Rev., 18:251-303.
Guillard, R. R. and Ryther, J. H. 1962. Studies on marine planktonic diatoms. 1. Cyclotella nana Hustedt and Detonula confervacaea (Cleve) Gran. Canadian Journal of Microbiology 8: 229-239
Haas, H., and J. C. Streibig., 1982. Changes in weed distribution patterns as a result of herbicide use and other agronomic factors. p. 57-80. In: H. M. LeBaron and J. Gressel (eds.), Herbicide resistance in plants. Wiley, New-York.
Hacham Y, Matityahu I, Schuster G, Amir R (2008) Overexpression of mutated forms of aspartate kinase and cystathionine gamma-synthase in tobacco leaves resulted in the high accumulation of methionine and threonine. Plant J 54: 260-271
Hedden, P., 1997. The oxidases of gibberellin biosynthesis: Their function and mechanism. Physiol. Plant. 101:709-719.
Hedden, P., and Y. Kamiya., 1997. Gibberellin biosynthesis: Enzymes, genes and their regulation. Ann. Rev. Plant Physiol. Plant Mol. Biol. 48: 431-460.
Helliwell, C. A., C. C. Sheldon, M. R. Olive, A. R. Walker, J. A. D. Zeevaart, W. J. Peacock and E. S. Dennis., 1998. Cloning of the Arabidopsis ent-kaurene oxidase gene GA3. Proc. Natl. Acad. Sci. U.S.A. 95:9019-9024.
Holm, L., J. Doll, E. Holm, J. Pancho, and J. Herberger., 1997. Worlds weeds: Natural histories and distributions. Wiley, New York.
Holt, J. S., 1988. Ecological and physiological characteristics of weeds. P. 7-23. In: M. A. Altieri and M. Liebman (eds.), Weed Management in Agroecosystems: Ecological Approaches. CRC Press, Inc. Boca Raton, Fla.
Hyatt, L. A., and A. S. Evans., 1998. Is decreased germination fraction associated with risk of sibling competition? OIKOS. 83: 29-35
Jorgensen, R. B., and B. Andersen., 1994. Spontaneous hybridization between oilseed rape (Brassica napus) and weedy B. campestris: a risk of growing genmodified oilseed rape. Am. J. Bot. 81: 1620-1626.
Kang, R. J. et al., 2005. Effects of co-expression of two higher plants genes ALD and TPI in Anabaena sp. PCC7120 on photosynthetic CO₂ fixation, Enzym. Microb. Technol. 36: 600-604′
Kareiva, P., I. M. Parker, and M. Pascual., 1996. Can we use experiments and models in predicting the invasiveness of genetically engineered organisms? Ecology 77:1670-1675.
Keeler, K. H., C. E. Turner, and M. R. Bollick., 1996. Movement of crop transgenes into wild plants. p. 303-330 In: S. O. Duke (ed.), Herbicide resistant crops: Agricultural, environmental, economic, regulatory, and technical aspects. CRC Press, Boca Raton.
Kerlan, M. C., A. M. Chevre, and F. Eber, F., 1993. Interspecific hybrids between a transgenic rapeseed (Brassica napus) and related species: cytogenetical characterization and detection of the transgene. Genome 36:1099-1106.
Khan, A. A., 1997. Quantification of seed dormancy: Physiological and molecular considerations. Hortsci. 32: 609-614.
Khrebtukova, I. and R. J. Spreitzer, 1996. Elimination of the Chlamydomonas gene family that encodes the small subunit of ribulose-1,5-bisphosphate carboxylaseoxygenase. Proc. Natl. Acad. Sci. USA 93: 13689-13693
Kimber, G., and E. R. Sears., 1987. Evolution of the genus Triticum and the origin of cultivated wheat. In: E. G. Heyne (ed.), Wheat and wheat improvement. Agronomy Monograph No. 13. ASA-SCCA-SSSA, Madison Wis., pages. 154-164.
Kindle K L (1990) High-frequency nuclear transformation of Chlamydomonas reinhardtii. Proceedings of the National Academy of Sciences of the United States of America 87: 1228
Kjellsson, G., V. Simonsen, and K. Ammann, (eds.)., 1994. Methods for risk assessment of transgenic plants. Vol. 2. Pollination, gene transfer and population impacts. Birkhaeuser, Base1.
Kling, J., 1996. Could transgenic supercrops one day breed superweeds? Science 274:180-181.
Kloppenburg, J., Jr., 1988. First the seeds: The political economy of plant biotechnology, Cambridge Univ. Press, Cambridge.
Koltunow, A. M., R. A. Bicknell, and A. M. Chaudhury., 1995. Apomixis: Molecular strategies for the generation of genetically-identical seeds without fertilization. Plant Physiol. 108:1345-1352.
Krimsky, S., and R. Wrubel., 1996. Agricultural biotechnology: Science, policy, and social issues. Univ. III. Press, Urbana.
Kusaba, S., M. Fukumoto, C. Honda, I. Yamaguchi, T. Sakamoto, and Y. Kano-Murakami., 1998. Decreased GA(1) content caused by the overexpression of OSH1 is accompanied by suppression of GA₂₀ and oxidase gene expression. Plant Physiol. 117:1179-1184.
Landbo, L., B. Andersen, and R. B. Jorgensen., 1996. Natural hybridization between oilseed rape and a wild relative: hybrids among seeds from weedy B. campestris. Hereditas 125:89-91.
Lange, T., 1998. Molecular biology of gibberellin synthesis. Planta 204:409-419.
Lange, T., S. Robatzek, and A. Frisse., 1997. Cloning and expression of gibberellin 2 β,3β-hydroxylas cDNA from pumpkin endosperm. Plant Cell 9:1459-1467.
Lee, J. W. L. Mets and E. Greenbaum, 2002. Improvement of photosynthetic CO2 fixation at high light intensity through reduction of chlorophyll antenna size. Applied Biochemistry and Biotechnology 98-100: 37-48.
Lefol, E., A. Fleury, and H. Darmency., 1996b. Gene dispersal from transgenic crops II. Hybridization between oilseed rape and the wild hoary mustard. Sex. Plant Reprod. 9:189-196.
Lefol, E., V. Danielou, and H. Darmency., 1996a. Predicting hybridization between transgenic oilseed rape and wild mustard. Field Crops Res. 45: 153-161.
Levy, A., 1985. A shattering-resistant mutant of Papaver bracteatum Lindl: characterization and inheritance. Euphytica 34: 811-815.
Li, B. L., and M. E. Foley., 1997. Genetic and molecular control of seed dormancy. Trends Plant Sci. 2:384-389.
Lin, S. Y., T. Sasaki, M. Yano., 1998. Mapping quantitative trait loci controlling seed dormancy and heading date in rice, Oryza sativa L., using backcross inbred lines. Theor. Appl. Genet. 96:997-1003.
Lincoln, J. and Fischer, R., 1988. Diverse mechanisms for the regulation of ethylene-inducible gene expression. Mol. Gen. Genet. 212:71-75.
Linder, C. R., 1998. Potential persistence of transgenes: seed performance of transgenic canola and wild X canola hybrids. Ecol. Appli. 8:1180-1195.
Ling-Hwa, T., and H. Morishima., 1997. Genetic characterization of weedy rices and the inference on their origins. Breeding Sci. 47:153-160
Love, S. L., 1994. Ecological risk of growing transgenic potatoes in the United States and Canada. Amer. Potato J. 71:647-658.
Lumbreras V, Stevens D R, Purton S (1998) Efficient foreign gene expression in Chlamydomonas reinhardtii mediated by an endogenous intron. Plant Journal 14: 441-447
Lundberg, S., P. Nilsson, and T. Fagerstrom., 1996. Seed dormancy and frequency dependent selection due to sib competition: The effect of age specific gene expression. J. Theor. Biol. 183:9-17.
Lutman, P. J. W. 1993. The occurrence and persistence of volunteer rapeseed (Brassica napus). Asp. Appl. Biol. 35:29-36.
Ma, W. M., L. Z. Wei, Q. X. Wang, D. J. Shi, H. B. Chen, 2007 Increased activity of the non-regulated enzymes fructose-1,6-bisphosphate aldolase and triosephosphate isomerase in Anabaena sp strain PCC 7120 increases photosynthetic yield, J. Appl. Phycol. 19: 207-213
Melis, A., J. Neidhardt, J. R. Benemann, 1998. Dunaliella salina (Chlorophyta) with small chlorophyll antenna sizes exhibit higher photosynthetic productivities and photon use efficiencies than normally pigmented cells, J. Appl. Phycol. 10: 515-525.
Metz, P. L. J., E. Jacobsen, J. P. Nap, A. Pereira, and W. J. Steikema., 1997. The impact of biosafety on the phosphinothricin-tolerance transgene in interspecific B. rapa x B. napus hybrids and their successive backcrosses. Theor. Appl. Gen. 95:442-450.
Mikkelsen, T. R., J. Jensen, and R. B. Jorgensen., 1996. Inheritance of oilseed rape (Brassica napus) RAPD markers in a backcross with progeny with Brassica campestris. Theor. Appl. Genet. 92:492-497.
Nishijima, T., N. Katsura, M. Koshioka, H. Yamazaki, M. Nakayama, H. Yamane, I. Yamaguchi, T. Yokota, N. Murofushi, N. Takahashi, and M. Nonaka., 1998. Effects of gibberellins and gibberellin-biosynthesis inhibitors on stem elongation and flowering of Raphanus sativus L. J. Jap. Soc. Hort. Sci. 67:325-330.
Ochman H, Gerber A S, Hartl D L (1988) Genetic Applications of an Inverse Polymerase Chain Reaction. Genetics 120: 621-623
Okamoto S, Ohmori M (2002) The Cyanobacterial PilT Protein Responsible for Cell Motility and Transformation Hydrolyzes ATP. Plant and Cell Physiology 43: 1127-1136
Pantone, D. J., and J. B. Baker., 1991. Weed-crop competition models and response-surface analysis of red rice competition in cultivated rice: A review. Crop Sci. 31:1105-1110.
Paterson, A. H., K. F. Schertz, Y-R. Lin, S-C. Liu and Y-L. Chang., 1995. The weediness of wild plants: Molecular analysis of genes influencing dispersal and persistence of johnsongrass, Sorghum halepense (L.) Pers. Proc. Natl. Acad. Sci. U.S.A. 92: 6127-6131.
Polle J E, Kanakagiri S D, Melis A (2003) tla1, a DNA insertional transformant of the green alga Chlamydomonas reinhardtii with a truncated light-harvesting chlorophyll antenna size. Planta 217: 49-59
Powell, M., 1997 Science in sanitary and phytosanitary dispute resolution. Discussion Paper 97-50, Resources for the Future, Washington, D.C.
Prakash, S., 1988. Introgression of resistance to shattering in Brassica napus from Brassica juncea through non-homologous recombination. Z. Pflanzenzuch. 101:167-168.
Price, J. S, R. N. Hobson, M. A. Nealle, and D. M. Bruce., 1996. Seed losses in commercial harvesting of oilseed rape. J. Agr. Engineer. Res. 65:183-191.
Regal, P. J., 1994. Scientific principles for ecologically based risk assessment of transgenic organisms. Molec. Ecol. 3:5-13.
Rissler, J. and M. Mellon., 1993. Perils amidst the promise—ecological risks of BD-HRCs in a global market. Union of Concerned Scientists, Cambridge Mass.
Robson, P. R. H., A. C. McCormac, A. S. Irvine, and H. Smith., 1996. Genetic engineering of harvest index in tobacco through overexpression of a phytochrome gene. Nature Biotech. 14:995-998.
Schaller, H., P. Bouvier-Naveo and P. Benveniste., 1998. Overexpression of an Arabidopsis cDNA encoding a sterol-C24-methyltransferase in tobacco modifies the ratio of 24-methyl cholesterol to sitosterol and is associated with growth reduction. Plant Physiol. 118:461-469.
Scheffler, J. A., R. Parkinson, and P. J. Dale., 1995. Evaluating the effectiveness of isolation distances for field plots of oilseed rape (Brassica napus) using a herbicide resistant transgene as a selectable marker. Plant Breed. 114: 317-321.
Schmidt G. W., Matlin K. S., and Chua N. (1977). A rapid procedure for selective enrichment of photosynthetic electron transport mutants. Proc. Natl. Acad. Sci. USA 74: 610-614
Sheehan, J., et al. (2004). A Look Back at the US Department of Energy's Aquatic Species Program Biodiesel from Algae; Close-Out Report, Island Press.
Simon, U., 1994. “Alko” the first seed-shattering resistant cultivar of meadow foxtail Alopecurus pratensis L. Acta Hort. 355: 143-146.
Sindel, B. M., 1997. Outcrossing of transgenes to weedy relatives. p. 43-81. In: G. D. McLean, P. M. Waterhouse G. Evans and M. J. Gibbs (eds.), Commercialisation of BD-HRCs: Risk, benefit and trade considerations. Coop. Res. Center for Plant Sci. and Bur. of Resource Sci., Canberra.
Sivan, A. and S. Arad 1998 Intraspecific transfer of herbicide resistance in the red microalga Porphyridium sp. (Rhodophyceae) via protoplast fusion. J. Phycol. 34, 706-711
Smith, M. W., S. Yamaguchi, T. Ait-Ali, and Y. Kamiya., 1998. The first step of gibberellin biosynthesis in pumpkin is catalyzed by at least two copalyl diphosphate synthases encoded by differentially regulated genes. Plant Physiol. 118: 1411-1419.
Smith, H., and G. C. Whitelam., 1997. The shade avoidance syndrome: Multiple responses mediated by multiple phytochromes. Plant Cell Environ. 20: 840-844.
Snow, A. A., P. Moran-Palma, L. H. Rieseberg, A. Wszelaki, and G. J. Seiler., 1998. Fecundity, phenology, and seed dormancy of F₁ wild-crop hybrids in sunflower (Helianthus annuus, Asteraceae). Amer. Jour. Bot. 85:794-801
Steber, C. M., S. E. Cooney, P. McCourt, 1998. Isolation of the GA-response mutant sly1 as a suppressor of ABI1-1 in Arabidopsis thaliana. Genetics 149:509-521
Tanner C A, Rompolas P, Patel-King R S, Gorbatyuk O, Wakabayashi K I, Pazour G J, King S M (2008) Three members of the LC8/DYNLL family are required for outer arm dynein motor function. Mol Biol Cell 19:3724-3734.
Thill, D. C., and C. A. Mallory-Smith., 1997. The nature and consequence of weed spread in cropping systems. Weed. Sci. 45:337-342.
Timmons, A. M., Y. M. Charters, J. W. Crawford, D. Burn, S. E. Scott, S. J. Dubbels, N. J. Wilson, A. Robertson, E. T. O'Brien, G. R. Squire and M. J. Wilkinson. 1. Risks from BD-HRCs. Nature 380: 487.
Torgersen, H., 1996. Risk assessment in transgenic plants: what can we learn from the ecological impacts of traditional crops? BINAS News 2: (3&4) (http://www.binas.unido.org/binas/News/96 issue34/risk.html)
Torii, K. U., T. W. McNellis, and X.-W. Deng., 1998. Functional dissection of Arabidopsis COP1 reveals specific roles of its three structural modules in light control of seedling development. EMBO J. 17:5577-5587.
Turner, C. E., 1988. Ecology of invasions by weeds. Weed Management in Agroecosystems: Ecological Approaches. pp. 41-54. In: M. A. Altieri and M. Liebman (eds.), Weed Management in Agroecosystems: Ecological Approaches. CRC Press, Inc. Boca Raton, Fla.
U, N. 1935. Genome analysis in Brassica with special reference to the experimental formation of B. napus and the peculiar mode of fertilization. Japan. J. Bot. 7: 389-452.
Van der Schaar, W., C. L. A. Blanco, K. M. Kloosterziel, R. C. Jansen, J. W. Van Ooijen, and M. Koornneef., 1997. QTL analysis of seed dormancy in Arabidopsis using recombinant inbred lines and MQM mapping. Heredity 79:, 190-200.
Van Etten J L, Burbank D E, Kuczmarski D, Meints R H (1983) Virus Infection of Culturable Chlorella-Like Algae and Development of a Plaque Assay. Science 219: 994-996
Vleeshouwers, L. M., 1988. The effect of seed dormancy on percentage and rate of germination in Polygonum persicaria, and its relevance for crop-weed interaction. Ann. Appl. Biol. 132:289-299.
Wan, J., T. Nakazaki, K. Kawaura, and H. Ikehashi., 1997. Identification of marker loci for seed dormancy in rice (Oryza sativa L.) Crop Sci. 37: 1759-1763.
Waters, S., 1996. The regulation of herbicide-resistant crops in Europe. p. 347-362. In: S. O. Duke (ed.), Herbicide resistant crops: Agricultural, environmental, economic, regulatory, and technical aspects. CRC Press, Boca Raton.
Webb, S. E., N. E. J. Appleford, P. Gaskin, and J. R. Lenton., 1998. Gibberellins in internodes and ears of wheat containing different dwarfing alleles Phytochemistry 47:671-677.
White, A. D., M. K. E. Owen, and R. G. Hartzler., 1998. Evaluation of common sunflower (Helianthus annuus L.) resistance to acetolactate synthase inhibiting herbicides. Weed Sci. Soc. Am. Abstr. 38:1120.
Whitton, J., D. E. Wolf, D. M. Arias, A. A. Snow, and L. H. Reiseberg., 1995. The persistence of cultivar alleles in wild populations of sunflowers five generations after hybridization. Theor. Appl. Genet. 95:35-40
Williams, M. E., 1995. Genetic engineering for pollen control. Trends Biotech. 13:344-349.
Williamson, M., 1993. Invaders, weeds, and risks from genetically manipulated organisms. Experientia 49:219-224.
Wilson W H, Joint I R, Carr N G, Mann N H (1993) Isolation and Molecular Characterization of Five Marine Cyanophages Propagated on Synechococcus sp. Strain WH7803. Applied and Environmental Microbiology 59: 3736
Yamaguchi, S., T. P. Sun, H. Kawaide, and Y. Kamiya., 1998. The GA₂ locus of Arabidopsis thaliana encodes ent-kaurene synthase of gibberellin biosynthesis. Plant Physiol. 116:1271-1278.
Young, B. A., 1991. Heritability of resistance to seed shattering in kleingrass., 1991. Crop Sci. 31:1156-1158.
Zemetra, R. S., J. Hansen, and C. A. Mallory-Smith., 1998. Potential for gene transfer between wheat (Triticum aestivum) and jointed goatgrass (Aegilops cylindrica). Weed Sci. 46: 313-317.
Zhu X, Galili G (2004) Lysine metabolism is concurrently regulated by synthesis and catabolism in both reproductive and vegetative tissues. Plant Physiol 135: 129-136

What is claimed is:

1. A method to mitigate effects of release of at least one advantageous genetically engineered trait of an alga or a cyanobacterium into natural ecosystems, said method comprising a step of transforming an alga or a cyanobacterium to express said at least one advantageous genetically engineered trait, and at least one mitigating genetic trait, wherein:
said at least one advantageous genetically engineered trait is encoded by at least one advantageous gene, and said at least one advantageous gene being operably linked with promoter sequences, and
said at least one mitigating genetic trait is encoded by at least one mitigating gene, said at least one mitigating gene being optionally operably linked with promoter sequences; and
said at least one advantageous gene and said at least one mitigating gene being introduced into the alga or cyanobacterium in tandem, whereby encoding sequences of the advantageous and mitigating genes remain genetically linked in the transgenic alga or cyanobacterium; and
said at least one mitigating gene further being desirable in or neutral to the transgenic alga or cyanobacterium when cultivated but rendering the transgenic alga or cyanobacterium incapable of establishing itself or its introgressed offspring in natural ecosystems.

2. The method of claim 1, wherein the alga is selected from the group of algal strains consisting of:
Nannochloropsis sp CS 246, Nannochloropsis oculata, Phaeodactylum tricornutum, Nannochloropsis salina, Pavlova lutheri CS182, Chlamydomonas reinhardtii, Isochrysis sp. Tetraselmis sp. and Chlorella sp.

3. The method of claim 1, wherein the cyanobacterium is selected from the group of cyanobacterial strains consisting of:
Synechococcus PC 7942, Synechococcus PCC7002, and Synechocystis PCC6803,

4. The method of claim 1, wherein the mitigating trait is selected from the group consisting of:
reduced content of ribulose 1,5 bis phosphate carboxylase/oxygenase (RUBISCO), reduced photosystem 2 antenna size, modified cilia or flagella formation or action, reduced carotene content in photosystems, modified content of cell wall polymers, and modified biosynthesis of storage polymers.

5. The method of claim 4, wherein the mitigating trait is reduced content of RUBISCO and the trait is conferred by an antisense oriented gene sequence encoding large or small subunit of RUBISCO or by RNAi cassette of the gene.

6. The method of claim 4, wherein the mitigating trait is reduced photosystem 2 antenna size, and the trait is conferred by an antisense oriented tla1 gene or by RNAi of the gene.

7. The method of claim 4, wherein the mitigating trait is modified cilia or flagella formation or action, and the trait is conferred by pliT-gene in antisense orientation.

8. The method of claim 4, wherein the mitigating trait is reduced carotene content of photosystems and the trait is conferred by a mutant pds-gene.

9. The method of claim 4, wherein the mitigating trait is modified content of cell wall polymers.

10. The method of claim 4, wherein the mitigating trait is modified biosynthesis of storage polymers.

11. The method of claim 10, wherein starch storage is reduced and nondegradable polysaccharide storage is enhanced.

12. The method of claim 11, wherein the non degradable polysaccharide is selected from the group consisting of inulin, levan and graminan.

13. The method of claim 10, wherein starch storage is reduced by a sta1 gene in antisense orientation or by an RNAi cassette of the gene.

14. The method of claim 12, wherein the non degradable polysaccharide is inulin and the trait is conferred by 1SST or 1FFT encoding sequences.

15. The method of claim 12, wherein the non degradable polysaccharide is levan and the trait is conferred by SacB or fif gene.

16. The method of claim 1, wherein the advantageous trait is selected from the group consisting of modified fatty acid composition, enhanced photosynthesis, increased methionine content, increased lysine content, herbicide resistance, mercury resistance, and virus resistance.

17. The method of claim 16, wherein the advantageous trait is modified fatty acid composition and the trait is conferred by a gene encoding delta(12)-fatty acid dehydrogenase.

18. The method of claim 16, wherein the advantageous trait is modified fatty acid composition and the trait is conferred by a gene encoding fatty acid desaturase.

19. The method of claim 16, wherein the advantageous trait is modified fatty acid composition and the trait is conferred by a gene encoding dithioesterase.

20. The method of claim 16, wherein the advantageous trait is enhanced photosynthesis and the trait is conferred by Tla1 gene.

21. The method of claim 16, wherein the advantageous trait is enhanced photosynthesis and the trait is conferred by a gene encoding Blue Fluorescent Protein (BFP).

22. The method of claim 16, wherein the advantageous trait is increased methionine content and the trait is conferred by a gene encoding high methionin 2S albumin.

23. The method of claim 16, wherein the advantageous trait is increased lysine content, and the trait is conferred by a gene encoding high lysine BHL8 protein.

24. The method of claim 16, wherein the advantageous trait is herbicide resistance and the trait is conferred by a gene selected from a group consisting of genes encoding 5-enolpyruvylshikimate-3-phosphate synthase (EPSPS), glyphosate oxidoreductase, acetolactate synthase, nitrilase, phospsphinothricin N-acetylatransferase, 4-hydroxyphenyl-puryvate dehydrogenase (HPPD) and protoporfyrinogen oxidase (PPO).

25. A genetic construct for mitigating effects of release of a genetically engineered genetic trait of an algae or cyanobacteria in to natural ecosystems, said construct comprising a first polynucleotide sequence encoding a desirable genetic trait and a second polynucleotide sequence encoding a mitigating genetic trait, wherein the first and the second polynucleotide sequences are covalently linked.

26. The genetic construct of claim 25, wherein the first polynucleotide sequence is operably linked to a promoter sequence.

27. The genetic construct of claim 26, wherein the first polynucleotide sequence is EPSPS coding sequence, the promoter sequence is RbcS promoter sequence, and the second polynucleotide sequence is tla1 encoding sequence in antisense orientation.

28. The genetic construct of claim 26, wherein the first polynucleotide sequence is virus CAPSID coding sequence, the promoter sequence is RbcS promoter sequence, and the second polynucleotide comprises RbcS RNAi cassette.

29. The genetic construct of claim 26, wherein the first polynucleotide sequence is virus CAPSID coding sequence, the promoter sequence is RbcS promoter sequence, and the second polynucleotide sequence is RbcS encoding sequence in antisense orientation operably linked to RbcS promoter sequence.

30. The genetic construct of claim 26, wherein the first polynucleotide sequence is mutant PDS coding sequence, the promoter sequence is hsp70A RbcS promoter sequence linked to 5′ end of rbcS2, and the second polynucleotide sequence is BHL8 coding sequence or 2S albumin protein coding sequence.

31. The genetic construct of claim 26, wherein the first polynucleotide sequence is mutant PDS coding sequence, the promoter sequence is RbcLS promoter sequence, and the second polynucleotide sequence is BHL8 coding sequence or 2S albumin protein coding sequence.

32. The genetic construct of claim 26, wherein the first polynucleotide sequence is BFP encoding sequence, the promoter sequence is RbcS promoter sequence, and the second polynucleotide sequence is PilT encoding sequence in antisense orientation operably linked RbcS promoter sequence.

33. A genetic construct for mitigating effects of release of a genetically engineered genetic trait of an alga or cyanobacterium in to natural ecosystems, said construct comprising a first cassette and a second cassette, said first cassette comprising a first and a second polynucleotide sequences operably linked to a first and a second promoter sequences and encoding a first and a second desirable genetic trait,
said second cassette comprising a polynucleotide sequence encoding a mitigating trait, said polynucleotide sequence being operably linked to a promoter sequence; and said first and second cassettes being covalently linked.

34. The genetic construct of claim 33, wherein the first polynucleotide sequence is merA coding sequence, first promoter sequence is RbcS promoter sequence, the second polynucleotide sequence is merB coding sequence, second promoter sequences is RbcS promoter sequence and the polynucleotide sequence encoding mitigating trait is sta1 or sta6 RNAi cassette.

35. A method to mitigate effects of release of at least one advantageous genetically engineered trait of an alga or a cyanobacterium into natural ecosystems, said method comprising steps of:
a. selecting a mutated alga or a cyanobacterium to express said at least one mitigating trait, and
b. transforming the mutated alga or cyanobacterium with said at least one advantageous trait, wherein said at least one advantageous genetically engineered trait is encoded by at least one advantageous gene, and said at least one advantageous gene being operably linked with promoter sequences, and said at least one mitigating trait being desirable in or neutral to the transformed alga or cyanobacterium when cultivated but rendering the alga or cyanobacterium incapable of establishing itself or its introgressed offspring in natural ecosystems.

36. The method of claim 35, wherein the mitigating trait is decreased antenna size and the advantageous trait is herbicide resistance.

37. The method of claim 36, wherein the mutated alga or cyanobacteria is transformed with an expression vector comprising HPPD resistance encoding gene sequence under control of rbcS2 promoter.

38. The method of claim 35, wherein the mitigating trait is impaired motility and the advantageous trait is herbicide resistance.

39. The method of claim 38, wherein the mutated alga is transformed with an expression vector comprising PPO herbicide resistance encoding gene sequence under control of rbcS2 promoter.

40. The method of claim 35, wherein the mutated alga is oda1-12 or tla-1 mutant of Chlamydomonas reinhardtii.

41. The method of claim 35, wherein the mutated cyanobacteria is PilT mutant.

42. A method to mitigate effects of release of at least one advantageous genetically engineered trait of an asexual, or non-conjugating alga or a cyanobacterium into natural ecosystems, said method comprising a step of transforming an alga or a cyanobacterium to express said at least one
advantageous genetically engineered trait, and at least one mitigating genetic trait, wherein:
said at least one advantageous genetically engineered trait is encoded by at least one advantageous gene, and said at least one advantageous gene being operably linked with promoter sequences, and
said at least one mitigating genetic trait is encoded by at least one mitigating gene, said at least one mitigating gene being optionally operably linked with promoter sequences; and
said at least one mitigating gene further being desirable in or neutral to the transgenic alga or cyanobacterium when cultivated but rendering the transgenic alga or cyanobacterium incapable to compete in natural ecosystems.


SEQUENCE LISTING (69 sequences)

SEQ ID NO: 1 - DNA, 1365 bp, Agrobacterium sp
  misc_feature (1)..(1365): EPSPS encoding polynucleotide sequence
atgagccacggcgccagcagccgccccgccaccgcccgcaagagcagcggcctgagcggcaccgtgcgcatccccggcgacaagagcatcagccaccgcagcttcatgttcggcggcctggccagcggcgagacccgcatcaccggcctgctggagggcgaggacgtgatcaacaccggcaaggccatgcaggccatgggcgcccgcatccgcaaggagggcgacacctggatcatcgacggcgtgggcaacggcggcctgctggcccccgaggcccccctggacttcggcaacgccgccaccggctgccgcctgaccatgggcctggtgggcgtgtacgacttcgacagcaccttcatcggcgacgccagcctgaccaagcgccccatgggccgcgtgctgaaccccctgcgcgagatgggcgtgcaggtgaagagcgaggacggcgaccgcctgcccgtgaccctgcgcggccccaagacccccacccccatcacctaccgcgtgcccatggccagcgcccaggtgaagagcgccgtgctgctggccggcctgaacacccccggcatcaccaccgtgatcgagcccatcatgacccgcgaccacaccgagaagatgctgcagggcttcggcgccaacctgaccgtggagaccgacgccgacggcgtgcgcaccatccgcctggagggccgcggcaagctgaccggccaggtgatcgacgtgcccggcgaccccagcagcaccgccttccccctggtggccgccctgctggtgcccggcagcgacgtgaccatcctgaacgtgctgatgaaccccacccgcaccggcctgatcctgaccctgcaggagatgggcgccgacatcgaggtgatcaacccccgcctggccggcggcgaggacgtggccgacctgcgcgtgcgcagcagcaccctgaagggcgtgaccgtgcccgaggaccgcgcccccagcatgatcgacgagtaccccatcctggccgtggccgccgccttcgccgagggcgccaccgtgatgaacggcctggaggagctgcgcgtgaaggagagcgaccgcctgagcgccgtggccaacggcctgaagctgaacggcgtggactgcgacgagggcgagaccagcctggtggtgcgcggccgccccgacggcaagggcctgggcaacgccagcggcgccgccgtggccacccacctggaccaccgcatcgccatgagcttcctggtgatgggcctggtgagcgagaaccccgtgaccgtggacgacgccaccatgatcgccaccagcttccccgagttcatggacctgatggccggcctgggcgccaagatcgagctgagcgacaccaaggccgcc

SEQ ID NO: 2 - DNA, 634 bp, Chlamydomonas reinhardtii
  misc_feature (1)..(634): Tla1 encoding polynucleotide sequence
ggaacctcgatgtcgtgttgactttgcgttacaaccgtgaagtatattagaactcatttgcctgccacaacctcagaccaagagacgcgcgaaaaactgacacgatgactttcagctgctccgctgaccaaaccgcgctcttaaagattcttgcacacgcggctaagtatccatcaaatagcgtgaatggtgtcctcgtcgggacagcgaaggagggcggctctgtcgaaatcctggacgcgattccactgtgtcacacgacgctgaccctggcgccagcactggagataggctcgcccaggtggagtcctacacgcatatcacgggcagcgtggcgattgtcctatctccagtgctggcgccagggtcagcgtcgtgtgacacagtggaatcgcgtccaggatttcgacagagccgccctccttcgctgtcccgacgaggacaccattcacgctatttgatggatacttagccgcgtgtgcaagaatctttaagagcgcggtttggtcagcggagcagctgaaagtcatcgtgtcagtttttcgcgcgtctcttggtctgaggttgtggcaggcaaatgagttctaatatacttcacggttgtaacgcaaagtcaacacgacatcgaggttcc

SEQ ID NO: 3 - DNA, 19 bp, artificial sequence
  chemically synthetized
tccccggcgacaagagcat

SEQ ID NO: 4 - DNA, 20 bp, artificial sequence
  chemically synthetized
aagagcgcggtttggtcagc

SEQ ID NO: 5 - DNA, 20 bp, artificial sequence
  chemically synthetized
caccgcatcgccatgagctt

SEQ ID NO: 6 - DNA, 20 bp, artificial sequence
  chemically synthetized
gctgaccaaaccgcgctctt

SEQ ID NO: 7 - DNA, 20 bp, artificial sequence
  chemically synthetized
atgggccaccaaaacgccgc

SEQ ID NO: 8 - DNA, 20 bp, artificial sequence
  chemically synthetized
cccactaactgtttggcttc

SEQ ID NO: 9 - DNA, 1338 bp, Arabidopsis thaliana
  misc_feature (1)..(1338): HPPD encoding polynucleotide sequence
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 10 - DNA, 32 bp, artificial sequence
  chemically synthetized
tctagactgcagcgccgtcattgccaagtcct

SEQ ID NO: 11 - DNA, 32 bp, artificial sequence
  chemically synthetized
ggatccaagcttaatgtagtcgacctgggcgg

SEQ ID NO: 12 - DNA, 1291 bp, Chlorella virus (PBCV-1)
  misc_feature (1)..(1291): CAPSID encoding polynucleotide sequence according to Chlamydomonas codon usage
atggccggcggcctgagccagctggtggcctacggcgcccaggacgtgtacctgaccggcaacccccagatcaccttcttcaagaccgtgtaccgccgctacaccaacttcgccatcgagagcatccagcagaccatcaacggcagcgtgggcttcggcaacaaggtgagcacccagatcagccgcaacggcgacctgatcaccgacatcgtggtggagttcgtgctgaccaagggcggcaacggcggcaccacctactaccccgccgaggagctgctgcaggacgtggagctggagatcggcggccagcgcatcgacaagcactacaacgactggttccgcacctacgacgccctgttccgcatgaacgacgaccgctacaactaccgccgcatgaccgactgggtgaacaacgagctggtgggcgcccagaagcgcttctacgtgcccctgatcttcttcttcaaccagacccccggcctggccctgcccctgatcgccctgcagtaccacgaggtgaagctgtacttcaccctggccagccaggtgcagggcgtgaactacaacggcagcagcgccatcgccggcgccgcccagcccaccatgagcgtgtgggtggactacatcttcctggacacccaggagcgcacccgcttcgcccagctgccccacgagtacctgatcgagcagctgcagttcaccggcagcgagaccgccacccccagcgccaccacccaggccagccagaacatccgcctgaacttcaaccaccccaccaagtacctggcctggaacttcaacaaccccaccaactacggccagtacaccgccctggccaacatccccggcgcctgcagcggcgccggcaccgccgccgccaccgtgaccacccccgactacggcaacaccggcacctacaacgagcagctggccgtgctggacagcgccaagatccagctgaacggccaggaccgcttcgccacccgcaagggcagctacttcaacaaggtgcagccctaccagagcatcggcggcgtgacccccgccggcgtgtacctgtacagcttcgccctgaagcccgccggccgccagcccagcggcacctgcaacttcagccgcatcgacaacgccaccctgagcctgacctacaagacctgcagcatcgacgccaccagccccgccgccgtgctgggcaacaccgagaccgtgaccgccaacaccgccaccctgctgaccgccctgaacatctacgccaagaactacaacgtgctgcgcatcatgagcggcatgg

SEQ ID NO: 13 - DNA, 27 bp, artificial sequence
  chemically synthetized
tctagaatggccgccgtcattgccaag

SEQ ID NO: 14 - DNA, 27 bp, artificial sequence
  chemically synthetized
tctagaacgagcgcctccatttacacg

SEQ ID NO: 15 - DNA, 1374 bp, Synechococcus virus (Syn9)
  misc_feature (1)..(1374): Capsid encoding plynucleotide  sequence
atgtctttccaaaacctccaagaaaagtgggcacccgttcttgagcacgattctctccccgagattggtgattcctacaagaaaggagttgtcgcacaacttcttgaaaaccaagaaaaagcaatcgcagaagagggcaagatcctcaccgaaactctgcaaaccactggttacactggtggcgatacagtaactggtcccgtagcaggtttcgaccctgttctgatcagcctgatccgccgctctatgcctcagctgatcgcttatgacatcgctggtgtgcagccgatgactggtcctactggactgatcttcgcaatgcgtaccaactatggcgcagagcgtaaccccgcagcagctggctacgatgaagcattcttcaacgagcccaacgctggtttctctggcggtcctggcgcatacgatcctggcgcaactggcgttaccaacgatgctgaaggcaccaaccctgcactcctcaacgattcccccgctggaacctacgagcaagcagacgacgcaactggcatgagcaccgctacagttgaagcactcgacgattccactgctaacacggcattccgtgagatgggtttctcgatcgagaaggtaactgtcacagcacgcgctcgcgccctgaaggcagaatacagcatcgagatggcacaagacctgaaggcaattcatggtctggatgctgagcaggagctcgctaacatccttagcactgagatcctcgctgaaatcaaccgtgaggttgtccgtaccatctacaccaacgctgttgcaggtgctcaaaacaacaccgctaccgctggtgtattcgacctcgacgttgactccaacggtcgctggtctgttgagaagtttaagggtctcctcttccaaatcgagcgtgatgccaatgctattggtcatcagactcgtcgcgggaagggcaacatcctcatctgctctgctgatgttgtttctgctctgggtatggctggtgtcctcgactacacccctgctctgaatggcaacaacggtctcgcaggtgttgatgacacctccagcaccctggttggcacccttaacggtcgtatcaaggtctacgttgatccctactctgcaaacgttgctgacaagcacttctacgttgcaggttataagggcaccagcccctatgacgcaggtctcttctactgcccctacgtccccctccagcaggttcgtgcaatcaaccctgacaccttccagcccaagatcggcttcaagactcgctacggcatggtctcgaatcccttcgctggcggtcttacccaaggcagcggtgctcttaccgtcaacgctaacaagtactaccgtcgcgtccaggttgctaacctcatgtga

SEQ ID NO: 16 - DNA, 24 bp, artificial sequence
  chemically synthetized
atgaaaactttacctaaagaaaag

SEQ ID NO: 17 - DNA, 19 bp, artificial sequence
  chemically synthetized
gtaacgggtttggttgggc

SEQ ID NO: 18 - DNA, 20 bp, artificial sequence
  chemically synthetized
atggccggcggcctgagcca

SEQ ID NO: 19 - DNA, 20 bp, artificial sequence
  chemically synthetized
aatgtagtcgacctgggcgg

SEQ ID NO: 20 - DNA, 20 bp, artificial sequence
  chemically synthetized
atggccggcggcctgagcca

SEQ ID NO: 21 - DNA, 20 bp, artificial sequence
  chemically synthetized
gtgtaaatggaggcgctcgt

SEQ ID NO: 22 - DNA, 20 bp, artificial sequence
  chemcially synthetized
atgtctttccaaaacctcca

SEQ ID NO: 23 - DNA, 20 bp, Artificial sequence
  Chemically synthetized
agcccaaccaaacccgttac

SEQ ID NO: 24 - DNA, 1740 bp, Hydrilla sp
  misc_feature (1)..(1740): PDS encoding polynucleotide sequence according to Chlamydomonas codon usage
atgaccgtggcccgcagcgtggtggccgtgaacctgagcggcagcctgcagaaccgctaccccgccagcagcagcgtgagctgcttcctgggcaaggagtaccgctgcaacagcatgctgggcttctgcggcagcggcaagctggccttcggcgccaacgccccctacagcaagatcgccgccaccaagcccaagcccaagctgcgccccctgaaggtgaactgcatggacttcccccgccccgacatcgacaacaccgccaacttcctggaggccgccgccctgagcagcagcttccgcaacagcgcccgccccagcaagcccctgcaggtggtgatcgccggcgccggcctggccggcctgagcaccgccaagtacctggccgacgccggccacatccccatcctgctggaggcccgcgacgtgctgggcggcaaggtggccgcctggaaggacgacgacggcgactggtacgagaccggcctgcacatcttcttcggcgcctaccccaacgtgcagaacctgttcggcgagctgggcatcaacgaccgcctgcagtggaaggagcacagcatgatcttcgccatgcccaacaagcccggcgagttcagccgcttcgacttccccgaggtgctgcccgcccccctgaacggcatctgggccatcctgaagaacaacgagatgctgacctggcccgagaaggtgcagttcgccatcggcctgctgcccgccatgatcggcggccagccctacgtggaggcccaggacggcctgaccgtgcaggagtggatgcgcaagcagggcgtgcccgaccgcgtgaacgacgaggtgttcatcgccatgagcaaggccctgaacttcatcaaccccgacgagctgagcatgcagtgcatcctgatcgccctgaaccacttcctgcaggagaagcacggcagcaagatggccttcctggacggcaacccccccgagcgcctgtgcaagcccatcgccgaccacatcgagagcctgggcggccaggtgatcctgaacagccgcatccagaagatcgagctgaacgccgacaagagcgtgaagcacttcgtgctgaccaacggcaacatcatcaccggcgacgcctacgtgttcgccacccccgtggacatcctgaagctgctgctgcccgaggactggaaggagatcagctacttcaagaagctggacaagctggtgggcgtgcccgtgatcaacgtgcacatctggttcgaccgcaagctgaagaacacctacgaccacctgctgttcagccgcagccccctgctgagcgtgtacgccgacatgagcgtgacctgcaaggagtactacaaccccaaccagagcatgctggagctggtgttcgcccccgccgagaagtggatcagctgcagcgacagcgagatcatcaacgccaccatgcaggagctggccaagctgttccccgacgagatcagcgccgaccagagcaaggccaagatcctgaagtaccacgtggtgaagaccccccgcagcgtgtacaagaccgtgcccgactgcgagccctgccgccccctgcagcgcagccccatcgagggcttctacctggccggcgactacaccaagcagaagtacctggccagcatggagggcgccgtgctgagcggcaagctgtgcgcccaggccatcgtgcaggactgcagcctgctggccagccgcgtgcagaagagcccccagaccctgaccatcgcc

SEQ ID NO: 25 - DNA, 204 bp, Barley
  misc_feature (1)..(204): BHL8 encoding polynucleotide sequence according to Chlamydomonas codon usage
atggccaagatgaagtgcacctggcccgagctggtggtgggcaagaccgtggagaaggccaagaagatgatcatgaaggacaagcccgaggccaagatcatggtgctgcccgtgggcaccaaggtgaccggcgagtggaagatggaccgcgtgcgcctgtgggtggacaagaaggacaagatcgccaagacccccaagtgcggc

SEQ ID NO: 26 - DNA, 438 bp, Brazil nut
  misc_feature (1)..(438): 2S albumin encoding polynucleotide sequence according to Chlamydomonas codon usage
atggccaagatcagcgtggccgccgccgccctgctggtgctgatggccctgggccacgccaccgccttccgcgccaccgtgaccaccaccgtggtggaggaggagaaccaggaggagtgccgcgagcagatgcagcgccagcagatgctgagccactgccgcatgtacatgcgccagcagatggaggagagcccctaccagaccatgccccgccgcggcatggagccccacatgagcgagtgctgcgagcagctggagggcatggacgagagctgccgctgcgagggcctgcgcatgatgatgatgcgcatgcagcaggaggagatgcagccccgcggcgagcagatgcgccgcatgatgcgcctggccgagaacatccccagccgctgcaacctgagccccatgcgctgccccatgggcggcagcatcgccggcttc

SEQ ID NO: 27 - DNA, 1740 bp, Hydrilla sp
  misc_feature (1)..(1740): PDS encoding polynucleotide sequence according to cyanobacteria codon usage
atgaccgtggcccgctctgtggtggccgtgaatctctctggctctctccaaaatcgctatcccgcctcttcttctgtgtcttgttttctcggcaaagaatatcgctgtaattctatgctcggcttttgtggctctggcaaactcgcctttggcgccaatgccccctattctaaaattgccgccaccaaacccaaacccaaactccgccccctcaaagtgaattgtatggattttccccgccccgatattgataataccgccaattttctcgaagccgccgccctctcttcttcttttcgcaattctgcccgcccctctaaacccctccaagtggtgattgccggcgccggcctcgccggcctctctaccgccaaatatctcgccgatgccggccacattcccattctcctcgaagcccgcgatgtgctcggcggcaaagtggccgcctggaaagatgatgatggcgattggtatgaaaccggcctccacattttttttggcgcctatcccaatgtgcaaaatctctttggcgaactcggcattaatgatcgcctccaatggaaagaacactctatgatttttgccatgcccaataaacccggcgaattttctcgctttgattttcccgaagtgctccccgcccccctcaatggcatttgggccattctcaaaaataatgaaatgctcacctggcccgaaaaagtgcaatttgccattggcctcctccccgccatgattggcggccaaccctatgtggaagcccaagatggcctcaccgtgcaagaatggatgcgcaaacaaggcgtgcccgatcgcgtgaatgatgaagtgtttattgccatgtctaaagccctcaattttattaatcccgatgaactctctatgcaatgtattctcattgccctcaatcactttctccaagaaaaacacggctctaaaatggcctttctcgatggcaatccccccgaacgcctctgtaaacccattgccgatcacattgaatctctcggcggccaagtgattctcaattctcgcattcaaaaaattgaactcaatgccgataaatctgtgaaacactttgtgctcaccaatggcaatattattaccggcgatgcctatgtgtttgccacccccgtggatattctcaaactcctcctccccgaagattggaaagaaatttcttattttaaaaaactcgataaactcgtgggcgtgcccgtgattaatgtgcacatttggtttgatcgcaaactcaaaaatacctatgatcacctcctcttttctcgctctcccctcctctctgtgtatgccgatatgtctgtgacctgtaaagaatattataatcccaatcaatctatgctcgaactcgtgtttgcccccgccgaaaaatggatttcttgttctgattctgaaattattaatgccaccatgcaagaactcgccaaactctttcccgatgaaatttctgccgatcaatctaaagccaaaattctcaaatatcacgtggtgaaaaccccccgctctgtgtataaaaccgtgcccgattgtgaaccctgtcgccccctccaacgctctcccattgaaggcttttatctcgccggcgattataccaaacaaaaatatctcgcctctatggaaggcgccgtgctctctggcaaactctgtgcccaagccattgtgcaagattgttctctcctcgcctctcgcgtgcaaaaatctccccaaaccctcaccattgcc

SEQ ID NO: 28 - DNA, 204 bp, Barley
  misc_feature (1)..(204): BHL8 encoding polynucleotide sequence according to cyanobacteria codon usage
atggccaaaatgaaatgtacctggcccgaactcgtggtgggcaaaaccgtggaaaaagccaaaaaaatgattatgaaagataaacccgaagccaaaattatggtgctccccgtgggcaccaaagtgaccggcgaatggaaaatggatcgcgtgcgcctctgggtggataaaaaagataaaattgccaaaacccccaaatgtggc

SEQ ID NO: 29 - DNA, 438 bp, brazil nut
  misc_feature (1)..(438): 2S albumin encoding poynucelotide sequence according to cyanobacteria codon usage
atggccaaaatttctgtggccgccgccgccctcctcgtgctcatggccctcggccacgccaccgcctttcgcgccaccgtgaccaccaccgtggtggaagaagaaaatcaagaagaatgtcgcgaacaaatgcaacgccaacaaatgctctctcactgtcgcatgtatatgcgccaacaaatggaagaatctccctatcaaaccatgccccgccgcggcatggaaccccacatgtctgaatgttgtgaacaactcgaaggcatggatgaatcttgtcgctgtgaaggcctccgcatgatgatgatgcgcatgcaacaagaagaaatgcaaccccgcggcgaacaaatgcgccgcatgatgcgcctcgccgaaaatattccctctcgctgtaatctctctcccatgcgctgtcccatgggcggctctattgccggcttt

SEQ ID NO: 30 - DNA, 20 bp, artificial sequence
  chemically synthetized
atgactgttgctaggtcggt

SEQ ID NO: 31 - DNA, 20 bp, Artificial sequence
  chemically synthetized
gcacttgggggtcttggcga

SEQ ID NO: 32 - DNA, 1605 bp, Amaranthus tuberculatus
  misc_feature (1)..(1605): PPO  encoding polynucleotide sequence
atggtaattcaatccattacccacctttcaccaaaccttgcattgccatcgccattgtcagtttcaaccaagaactacccagtagctgtaatgggcaacatttctgagcgggaagaacccacttctgctaaaagggttgctgttgttggtgctggagttagtggacttgctgctgcatataagctaaaatcccatggtttgagtgtgacattgtttgaagctgattctagagctggaggcaaacttaaaactgttaaaaaagatggttttatttgggatgagggggcaaatactatgacagaaagtgaggcagaggtctcgagtttgatcgatgatcttgggcttcgtgagaagcaacagttgccaatttcacaaaataaaagatacatagctagagacggtcttcctgtgctactaccttcaaatcccgctgcactactcacgagcaatatcctttcagcaaaatcaaagctgcaaattatgttggaaccatttctctggagaaaacacaatgctactgaactttctgatgagcatgttcaggaaagcgttggtgaattttttgagcgacattttgggaaagagtttgttgattatgttatcgacccttttgttgcgggtacatgtggtggagatcctcaatcgctttccatgcaccatacatttccagaagtatggaatattgaaaaaaggtttggctctgtgtttgctggactaattcaatcaacattgttatctaagaaggaaaagggtggagaaaatgcttctattaagaagcctcgtgtacgtggttcattttcatttcaaggtggaatgcagacacttgttgacacaatgtgcaaacagcttggtgaagatgaactcaaactccagtgtgaggtgctgtccttgtcatataaccagaaggggatcccctcattagggaattggtcagtctcttctatgtcaaataataccagtgaagatcaatcttatgatgctgtggttgtcactgctccaattcgcaatgtcaaagaaatgaagattatgaaatttggaaatccattttcacttgactttattccagaggtgacgtacgtacccctttccgttatgattactgcattcaaaaaggataaagtgaagagacctcttgagggcttcggagttcttatcccctctaaagagcaacataatggactgaagactcttggtactttattttcctccatgatgtttcctgatcgtgctccatctgacatgtgtctctttactacatttgtcggaggaagcagaaatagaaaacttgcaaacgcttcaacggatgaattgaagcaaatagtttcttctgaccttcagcagctgttgggcactgaggacgaaccttcatttgtcaatcatctcttttggagcaacgcattcccattgtatggacacaattacgattctgttttgagagccatagacaagatggaaaaggatcttcctggatttttttatgcaggtaaccataagggtggactttcagtgggaaaagcgatggcctccggatgcaaggctgcggaacttgtaatatcctatctggactctcatatatacgtgaagatggatgagaagaccgcgtaa

SEQ ID NO: 33 - DNA, 1107 bp, Synechococcus sp. PCC 7002
  misc_feature (1)..(1107): PilT encoding polynucleotide sequence
atggattacatgatcgaagacctcatggagcaactcgtagaaatgggcggctccgatatgcacatccaggccggtgcccccgtttacttccgagtcagtgggaaacttggccccattaatgacgagcccctctccgcccaagatgcccaaaagctcatcttcagtatgctcaacaacacccaacgcaaagacctcgagcaaaattgggaactagactgttcctatggcgtgaaaggactcgcgcgcttccgggtcaatgtctacaaagaacggggctgttatgccgcttgcctgcgggccttgtcctcgaaaattcccaactttgatcaactcggtttacccgatattgtgcgggaaatggccgagcgtccccggggtttggtgctggttacgggccaaacgggttccgggaaaaccaccaccatggctgcgatgttggatttgatcaaccgcacccgtgccgaacatatcctcaccgtcgaagaccccattgaatacgttttcccaaaccacaaaagcctcttccaccaacggcaaaagggagaagacacgaaaagctttgccaatgcgctccgggccgcgctccgggaagatccagacatcatcctcgtcggggaaatgcgggatctcgaaaccatctccttggctatctccgccgctgaaactgggcacttggttttcgggacgctccacaccaactccgccgccagcaccatcgaccggatgttagatgtgttcccgccgattcaacagccccaaattcgggcgatgctctccaactctctcttggctgtttttagtcaatgtctagtgaaaaaagcgaatccgaaacccggtgaattcgggcgctctatggcccaagaaattatggttgtcaccccggcgatcgccaacctgattcgtgaaggaaaaagcgcccaggtctactcagcaatccaaacgggaatgaaactcggcatgcaaaccatggagcaagccctggcgggtttagtggccacaggcaccgttacctttgaagaagccctgtcgaagagtggtaaacccgatgaactacaacgactcgttgggggagccttgggggctagcccaaccgcaaaacgtcgctaa

SEQ ID NO: 34 - DNA, 1602 bp, Amaranthus tuberculatus
  misc_feature (1)..(1602): PPO encoding nucleotide sequence according to Synechococcus codon usage
atggtgatccaaagcattacccacctatctcccaacttggccctgcccagccccctttctgtgagtacgaagaattatcccgtagccgtgatgggtaacatttcggaacgggaagagcccacctctgccaaacgagtggccgttgtaggggcgggtgtaagcggccttgctgccgcgtataaactgaaaagtcatggactcagcgttaccttgttcgaagcggatagccgggccggaggcaagctcaaaaccgtgaaaaaggatgggtttatctgggatgaaggtgctaacaccatgaccgagtcggaagcagaagtttcttcccttattgacgatttgggtctccgcgagaagcaacagcttcccatcagtcaaaacaaacggtatatcgctcgcgatgggctcccagtcctgctaccgtcgaatcccgctgccttgctcacttctaatatcttaagcgctaaaagtaaattgcagattatgttagaaccgtttctctggcgcaaacataatgcaactgaattaagcgacgagcacgttcaagaatccgtcggtgagtttttcgaacgacacttcggtaaggaattcgtcgattacgtcattgatccattcgtcgctggcacatgtggcggggatccgcagtccctatcaatgcatcacacgtttccagaagtctggaatattgaaaaacgctttggtagtgtgtttgcaggcttaatccagagcacattactcagtaaaaaggaaaagggcggtgaaaacgcgtccattaaaaagccgcgggtgcgtgggtccttctcgtttcaagggggcatgcaaaccctggtggatacgatgtgtaaacaactcggtgaggacgaactgaaattgcagtgcgaagtcttgagcttaagttataaccagaagggcattccttctctcgggaattggtctgtctcgtcaatgtccaataacacgagtgaggatcaatcttatgacgcggtggtcgttactgcccctattcgtaatgtgaaggaaatgaagattatgaaatttggtaatcctttttccttagactttattccagaggtaacatacgttccgctctccgtgatgatcaccgcctttaaaaaggacaaggttaaacgccccctggaaggatttggtgtcctgatcccgtctaaagaacagcataacggtttaaaaaccctcgggacgcttttttcctctatgatgtttcccgaccgggcaccctcggatatgtgcctctttaccacatttgttggcgggagtagaaatcgtaaactggccaatgcctccaccgatgaactcaaacaaatcgtgagctccgatctgcaacaactcttgggcactgaagatgaaccctcatttgtgaatcatctattctggagtaatgccttccctctttacggacacaactacgatagcgttttgcgcgcgatcgacaagatggaaaaagatctccccggatttttctacgcggggaatcacaaaggcggcctgtcagtcggcaaagcgatggcgagtggctgtaaagccgcagaattggtgattagttatctagattcgcatatttatgtgaaaatggatgaaaaaaccgcc

SEQ ID NO: 35 - DNA, 20 bp, artificial sequence
  chemically synthetized
atggattacatgatcgaaga

SEQ ID NO: 36 - DNA, 20 bp, artificial sequence
  chemically synthtetized
gcgacgttttgcggttgggc

SEQ ID NO: 37 - DNA, 20 bp, artificial sequence
  chemically synthetized
atggtaattcaatccattac

SEQ ID NO: 38 - DNA, 20 bp, artificial sequence
  chemically synthetized
cggtcttctcatccatcttc

SEQ ID NO: 39 - DNA, 20 bp, artificial sequence
  chemically synthetized
ctggactctcatatatacgt

SEQ ID NO: 40 - DNA, 20 bp, artificial sequence
  chemically synthetized
caaccgcaaaacgtcgctaa

SEQ ID NO: 41 - DNA, 33 bp, artificial sequence
  chemically synthetized
gctctagagcatgctgttaatggcgacgcctgg

SEQ ID NO: 42 - DNA, 34 bp, artificial sequence
  chemically synthetized
gcggatccaagcttgaaccactccttgtcggtgg

SEQ ID NO: 43 - DNA, 2156 bp, Helianthus tuberosus
  misc_feature (1)..(2156): SST encoding polynucleotide sequence
ggcacgagaaaaaaccctccctcaggccaccacatgatggcttcatccaccaccaccacccctctcattctccatgatgaccctgaaaacctcccagaactcaccggttctccgacaactcgtcgtctatccatcgcaaaagtgctttcggggatccttgtttcggttctggttataggtgctcttgttgctttaatcaacaaccaaacatatgaatccccctcggccaccacattcgtaactcagttgccaaatattgatctgaagcgggttccaggaaagttggattcgagtgctgaggttgaatggcaacgatccacttatcattttcaacccgacaaaaatttcattagcgatcctgatggcccaatgtatcacatgggatggtatcatctattttatcagtacaaccctcaatctgccatctggggcaacatcacatggggccactcggtatcgaaagacatgatcaactggttccatctccctttcgccatggttcctgaccattggtacgacatcgaaggtgtcatgacgggttcggctacagtcctccctaatggtcaaatcatcatgctttactcgggcaacgcgtatgatctctcccaagtacaatgcttggcgtacgctgtcaactcgtcggatccacttcttatagagtggaaaaaatatgaaggtaaccctgtcttactcccaccaccaggagtaggctacaaggactttcgggacccatccacattgtggtcgggccctgatggtgaatatagaatggtaatggggtccaagcacaacgagactattggctgtgctttgatttaccataccactaattttacgcattttgaattgaaagaggaggtgcttcatgcagtcccacatactggtatgtgggaatgtgttgatctttacccggtgtccaccgtacacacaaacgggctggacatggtggataacgggccaaatgttaagtacgtgttgaaacaaagtggggatgaagatcgccatgattggtatgcaattggaagttacgatatagtgaatgataagtggtacccagatgacccggaaaatgatgtgggtatcggattaagatatgattttggaaaattttatgcgtccaagacgttttatgaccaacataagaagaggagagtcctttggggctatgttggagaaaccgatccccaaaagtatgacctttcaaagggatgggctaacattttgaatattccaaggaccgtcgttttggacctcgaaactaaaaccaatttgattcaatggccaatcgaggaaaccgaaaaccttaggtcgaaaaagtatgatgaatttaaagacgtcgagcttcgacccggggcactcgttccccttgagataggcacagccacacagttggatatagttgcgacattcgaaatcgaccaaaagatgttggaatcaacgctagaggccgatgttctattcaattgcacgactagtgaaggctcggttgcaaggagtgtgttgggaccgtttggtgtggtggttctagccgatgcccagcgctccgaacaacttcctgtatacttctatatcgcaaaagatattgatggaacctcacgaacttatttttgtgccgacgaaacaagatcatccaaggatgtaagcgtagggaaatgggtgtacggaagcagtgttcctgtcctcccaggcgaaaagtacaatatgaggttattggtggatcattcgatagtagagggatttgcacaaaacgggagaaccgtggtgacatcaagagtgtatccaacaaaggcgatctacaacgctgcgaaggtgtttttgttcaacaacgcgactggaatcagtgtgaaggcgtcgatcaagatctggaagatgggggaagcagaactcaatcctttccctcttcctgggtggactttcgaactttgatggttatattttggaccctatatatgtgttattatcatgatggttatattttggaccctatatatgtgttattatcatgaagcataagtttggactggagggggtattattgtaattttatatgcatgttctattacttgtgaggttatagtatgtaattaaattattatatactatatcaatttctaataaaaaaaaaaaaaaaaaaaaaaaaaaaaaaaaaaaaaaaa

SEQ ID NO: 44 - DNA, 2036 bp, Helianthus tuberosus
  misc_feature (1)..(2036): FFT encoding polynucleotide sequence
gggacgagtaccagtccagtcagtcaccatgcaaacccctgaaccctttacagaccttgaacatgaaccccacacacccctactggaccaccaccacaacccaccaccacaaaccaccacaaaacctttgttcaccagggttgtgtccggtgtcacctttgttttattcttctttggtttcgctatcgtattcattgttctcaaccaacagaattcttctgttcgtatcgtcaccaattcggagaaatcttttataaggtattcgcagaccgatcgcttgtcgtgggaacggaccgcttttcattttcagcctgccaagaattttatttacgatccagatggtcagttgtttcacatgggctggtaccatatgttctatcaatacaacccatacgcaccggtttggggcaatatgtcatggggtcactcagtgtccaaagacatgatcaactggtacgagctgccagtcgctatggtcccgaccgaatggtatgatatcgagggcgtcttatccgggtctaccacggtccttccaaacggtcagatctttgcattgtatactgggaacgctaatgatttttcccaattacaatgcaaagctgtacccgtaaacttatctgacccgcttcttattgagtgggtcaagtatgaggataacccaatcctgtacactccaccagggattgggttaaaggactatcgggacccgtcaacagtctggacaggtcccgatggaaagcataggatgatcatgggaactaaacgtggcaatacaggcatggtacttgtttactataccactgattacacgaactacgagttgttggatgagccgttgcactctgttcccaacaccgatatgtgggaatgcgtcgacttttacccggtttcgttaaccaatgatagtgcacttgatatggcggcctatgggtcgggtatcaaacacgttattaaagaaagttgggagggacatggaatggattggtattcaatcgggacatatgacgcgataaatgataaatggactcccgataacccggaactagatgtcggtatcgggttacggtgcgattacgggaggttttttgcatcaaagagtctttatgacccattgaagaaaaggaggatcacttggggttatgttggagaatcagatagtgctgatcaggacctctctagaggatgggctactgtttataatgttggaagaacaattgtactagatagaaagaccgggacccatttacttcattggcccgttgaggaagtcgagagtttgagatacaacggtcaggagtttaaagagatcaagctagagcccggttcaatcattccactcgacataggcacggctacacagttggacatagttgcaacatttgaggtggatcaagcagcgttgaacgcgacaagtgaaaccgatgatatttatggttgcaccactagcttaggtgcagcccaaaggggaagtttgggaccatttggtcttgcggttctagccgatggaaccctttctgagttaactccggtttatttctatatagctaaaaaggcagatggaggtgtgtcgacacatttttgtaccgataagctaaggtcatcactagattatgatggggagagagtggtgtatgggggcactgttcctgtgttagatgatgaagaactcacaatgaggctattggtggatcattcgatagtggaggggtttgcgcaaggaggaaggacggttataacatcaagggcgtatccaacaaaagcgatatacgaacaagcgaagctgttcttgttcaacaacgccacaggtacgagtgtgaaagcatctctcaagatttggcaaatggcttctgcaccaattcatcaataccctttttaattaccggctatcgctatcctttttgttattggtatttatgtatcttaattttcttttaaacctttttatttgataaatattagttcttgttattgtgcttctagtaataaatgaatggtgttatgggaaaaaaaaaaaaaaaaaaaaaaaaaaaaaaaaa

SEQ ID NO: 45 - DNA, 20 bp, artificial sequence
  chemically synthetized
gtgtgttgttggcaatcgag

SEQ ID NO: 46 - DNA, 20 bp, artificial sequence
  chemically synthetized
ctagattaccgtgccgtcgg

SEQ ID NO: 47 - DNA, 20 bp, artificial sequence
  chemically synthetized
tctcatcgcattgcgctgca

SEQ ID NO: 48 - DNA, 20 bp, artificial sequence
  chemically synthetized
ccaactttcctggaacccgc

SEQ ID NO: 49 - DNA, 20 bp, artificial sequence
  chemically synthetized
cacgtttagttcccatgatc

SEQ ID NO: 50 - DNA, 20 bp, artificial sequence
  chemically synthetized
caagcgtggaacacatctac

SEQ ID NO: 51 - DNA, 735 bp, artificial sequence
  chemically synthetized
atgtctaaaggtgaagaattattcactggtgttgtcccaattttggttgaattagatggtgatgttaatggtcacaaattttctgtctccggtgaaggtgaaggtgatgctacgtacggtaaattgaccttaaaatttatttgtactactggtaaattgccagttccatggccaaccttagtaactactttgagccatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaacttcaactctcacaatatatacatcatggctgacaaacaaaagaatggtatcaaagtgaacttcaaaattagacacaacattgaagatggttctgttcaattagctgaccattatcaacaaaatactccaattggtgatggtccagtcttgttaccagacaaccattacttatccacccaatcagccttatccaaagatccaaacgaaaagagagaccacatggtcctgttagaatttaggactgctgctggtattacccatggtatggatgaattgtacaaacaccaccaccaccaccactaa

SEQ ID NO: 52 - protein, 244 aa, artificial sequence
  chemically synthetized
MSKGEELFTGVVPILVELDGDVNGHKFSVSGEGEGDATYGKLTLKFICTTGKLPVPWPTLVTTLSHGVQCFSRYPDHMKQHDFFKSAMPEGYVQERTIFFKDDGNYKTRAEVKFEGDTLVNRIELKGIDFKEDGNILGHKLEYNFNSHNIYIMADKQKNGIKVNFKIRHNIEDGSVQLADHYQQNTPIGDGPVLLPDNHYLSTQSALSKDPNEKRDHMVLLEFRTAAGITHGMDELYKHHHHHH

SEQ ID NO: 53 - DNA, 732 bp, artificial
  chemically synthetized
atgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgaccaccctgagccacggcgtgcagtgcttcagccgctaccccgaccacatgaagcagcacgacttcttcaagagcgccatgcccgagggctacgtgcaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggccacaagctggagtacaacttcaacagccacaacatctacatcatggccgacaagcagaagaacggcatcaaggtgaacttcaagatccgccacaacatcgaggacggcagcgtgcagctggccgaccactaccagcagaacacccccatcggcgacggccccgtgctgctgcccgacaaccactacctgagcacccagagcgccctgagcaaggaccccaacgagaagcgcgaccacatggtgctgctggagttccgcaccgccgccggcatcacccacggcatggacgagctgtacaagcaccaccaccaccaccac

SEQ ID NO: 54 - protein, 244 aa, artificial; identical to SEQ ID NO: 52
  Synthetic Construct

SEQ ID NO: 55 - DNA, 732 bp, artificial sequence
  chemically synthetized
atgtctaaaggcgaagaactctttaccggcgtggtgcccattctcgtggaactcgatggcgatgtgaatggccacaaattttctgtgtctggcgaaggcgaaggcgatgccacctatggcaaactcaccctcaaatttatttgtaccaccggcaaactccccgtgccctggcccaccctcgtgaccaccctctctcacggcgtgcaatgtttttctcgctatcccgatcacatgaaacaacacgatttttttaaatctgccatgcccgaaggctatgtgcaagaacgcaccattttttttaaagatgatggcaattataaaacccgcgccgaagtgaaatttgaaggcgataccctcgtgaatcgcattgaactcaaaggcattgattttaaagaagatggcaatattctcggccacaaactcgaatataattttaattctcacaatatttatattatggccgataaacaaaaaaatggcattaaagtgaattttaaaattcgccacaatattgaagatggctctgtgcaactcgccgatcactatcaacaaaatacccccattggcgatggccccgtgctcctccccgataatcactatctctctacccaatctgccctctctaaagatcccaatgaaaaacgcgatcacatggtgctcctcgaatttcgcaccgccgccggcattacccacggcatggatgaactctataaacaccaccaccaccaccac

SEQ ID NO: 56 - DNA, 567 bp, Chlamydomonas reinhardtii
  misc_feature (1)..(567): rbcA2B encoding polynucleotide sequence
atggccgccgtcattgccaagtcctccgtctccgcggccgtggcccgcccggcccgctccagcgtgcgccccatggccgcgctgaagcccgccgtcaaggccgcccccgtggctgccccggctcaggccaaccagatgatggtctggaccccggtcaacaacaagatgttcgagaccttctcctacctgccccccctgagcgacgagcagatcgccgcccaggtcgactacattgtcgccaacggctggatcccctgcctggagttcgctgagtcggacaaggcctacgtgtccaacgagtcggccatccgcttcggcagcgtgtcttgccgacaaccgctactggaccatgtggaagctgcccatgttcggctgccgcgaccccatgcaggtgctgcgcgagatcgtcgcctgcaccaaggccttccccgatgcctacgtgcgcctggtggccttcgacaaccagaagcaggtgcagatcatgggcttcctggtccagcgccccaagtctgcccgcgactggcagcccgccaacaagcgctccgtgtaaatggaggcgctcgttga

SEQ ID NO: 57 - DNA, 20 bp, artificial sequence
  chemically syntehtized
atggattacatgatcgaaga

SEQ ID NO: 58 - DNA, 20 bp, artificial sequence
  pilT reverse primer
gcgacgttttgcggttgggc

SEQ ID NO: 59 - DNA, 20 bp, artificial sequence
  chemically synthetized
atgagcaagggcgaggagct

SEQ ID NO: 60 - DNA, 20 bp, artificial sequence
  chemically synthetized
gtggtggtggtggtggtgct

SEQ ID NO: 61 - DNA, 20 bp, artificial sequence
  chemically synthetized
aacaccaccaccaccaccac

SEQ ID NO: 62 - DNA, 20 bp, artificial sequence
  chemically syntehtized
caaccgcaaaacgtcgctaa

SEQ ID NO: 63 - DNA, 1695 bp, E. coli
  misc_feature (1)..(1695): merA encoding polynucleotide sequence
atgagcactctcaaaatcaccggcatgacttgcgactcgtgcgcagtgcatgtcaaggacgccctggagaaagtgcccggcgtgcaatcagcggatgtctcctacgccaagggcagcgccaagctcgccattgaggtcggcacgtcacccgacgcgctgacggccgctgtagctggactcggttatcgggccacgctggccgatgccccctcagtttcgacgccgggcggattgctcgacaagatgcgcgatctgctgggcagaaacgacaagacgggtagcagcggcgcattgcatatcgccgtcatcggcagcggcggggccgcgatggcagcggcgctgaaggccgtcgagcaaggcgcacgtgtcacgctgatcgagcgcggcaccatcggcggcacctgcgtcaatgtcggttgtgtgccgtccaagatcatgatccgcgccgcccatatcgcccatctgcgccgggaaagcccgttcgatggcggcatcgccgctaccacgccgaccatccagcgcacggcgctgctggcccagcagcaggcccgcgtcgatgaactgcgccacgccaagtacgaaggcatcttggagggcaatccggcgatcactgtgctgcacggctccgcccgctttaaggacaatcgcaacctgatcgtgcaactcaacgacggcggcgagcgcgtggtggcattcgaccgctgcctgatcgccaccggcgcgagcccggccgtgccgccgattcccggcctgaaagacactccgtactggacttccactgaagcgctggtcagcgagacgattcctaagcgcctggccgtgattggctcatcagtggtggcgctggagctggcgcaggcgttcgcccgactcggagcgaaggtgacgatcctggctcgcagcacgctgttcttccgcgaagacccagctataggcgaagccgtcacggccgcattccgcatggagggcatcgaggtgagggaacacacccaggccagccaggtcgcgtatatcaatggtgaaggggacggcgaattcgtgctcaccacggcgcacggcgaactgcgcgccgacaagctgctggtcgccaccggccgcgcgcccaacacacgcaagctggcactggatgcgacgggcgtcacgctcaccccgcaaggcgctatcgtcatcgaccccggcatgcgtacaagcgtggaacacatctacgccgcaggcgactgcaccgaccagccgcagttcgtctatgtggcggcagcggccggcactcgcgccgcgatcaacatgaccggcggtgacgcggccctgaacctgaccgcgatgccggccgtggtgttcaccgacccgcaagtggcgaccgtaggctacagcgaggcggaagcgcaccatgacggcatcaaaactgatagtcgcacgctaacgctggacaacgtgccgcgcgcgctcgccaacttcgacacgcgcggcttcatcaaactggtggttgaagaaggcagcggacgactgatcggcgtgcaggcagtggccccggaagcgggcgaactgatccagacggccgcactggcgattcgcaaccggatgacggtgcaggaactggccgaccagttgttcccctacctgacgatggtcgaagggttgaagctcgcggcgcagaccttcaacaaggatgtgaagcagctttcctgctgcgccgggtga

SEQ ID NO: 64 - DNA, 639 bp, E.coli
  CDS (1)..(639): merB encoding polynucleotide sequence
atgaagctcgccccatatattttagaacttctcacttcggtcaatcgtaccaatggtactgcggatctcttggtcccgctactgcgggaactcgccaaggggcgtccggtttcacgaacgacacttgccgggattctcgactggcccgctgagcgagtggccgccgtactcgaacaggccaccagtaccgaatatgacaaagatgggaacatcatcggctacggcctcaccttgcgcgagacttcgtatgtctttgaaattgacgaccgccgtctgtatgcctggtgcgcgctggacaccttgatatttccggcgctgatcggccgtacagctcgcgtctcatcgcattgcgctgcaaccggagcaccggtttcactcacggtttcacccagcgagatacaggctgtcgaacctgccggcatggcggtgtccttggtattgccgcaggaagcagccgacgttcgtcagtccttctgttgccatgtacatttctttgcatctgtcccgacggcggaagactgggcctccaagcatcaaggattggaaggattggcgatcgtcagtgtccacgaggctttcggcttgggccaggagtttaatcgacatctgttgcagaccatgtcatctaggacaccgtga

SEQ ID NO: 65 - protein, 212 aa, E.coli
MKLAPYILELLTSVNRTNGTADLLVPLLRELAKGRPVSRTTLAGILDWPAERVAAVLEQATSTEYDKDGNIIGYGLTLRETSYVFEIDDRRLYAWCALDTLIFPALIGRTARVSSHCAATGAPVSLTVSPSEIQAVEPAGMAVSLVLPQEAADVRQSFCCHVHFFASVPTAEDWASKHQGLEGLAIVSVHEAFGLGQEFNRHLLQTMSSRTP

SEQ ID NO: 66 - DNA, 336 bp, Synechococcus PCC700
  misc_feature (1)..(336): rbcS2B encoding polynucleotide sequence
atgaaaactttacctaaagaaaagcgttacgaaactctttcttacttgccccccctcagcgaccagcaaatcgctcgccaagtccagtacatgatggatcaaggctatattcctggtatcgagttcgaaaaagatccgactcctgaactccaccactggacactgtggaagctgccccttttcaacgcaagctctgctcaagaagtactcaacgaagtgcgtgagtgccgtagtgaatattctgactgctacatccgtgttgttggtttcgacaacatcaagcagtgccaaaccgttagcttcatcgtttacaagcccaaccaaacccgttactaa

SEQ ID NO: 67 - DNA, 1053 bp, Chlamydomonas reinhardtii
  misc_feature (1)..(1053): sta1 partial gDNA of C. reinhardtii, includes exons 2,3,4 and introns 2,3
atgttaatggcgacgcctggttttgggtcgtgtgctgtccgggggggtcagggttgcgaggccgcggccagctcgagacgcgtcgaggagtcgtgcccagcctccttatgcgctggctgctgctatgtgctgtcagaccgggagcaggaccgacagccacgcggcgaagtctgctcatgtgcaccagtggcatagggagattcttgcgcgtgtggcgacgggcaggggctgcgtgccaccccgtaccaacgctgccgcacccggcgcctcttcatctcttctggctccacagcaccgaaccccaatccttaacctccgtgaccacatcagctcttgtgaccctgaccctcgccccatgagagcattacggctcttgtcacggtcaaacccgccatcgccgctacccccgctccacctccctgcaccccacccctctcacggcctcactcccccctcaccccctcgcccaccatccatacacagccatcattctgggtggcggcgccggcacccgcctgttcccgctgaccaagtcgcgcgccaagccggccgtgcccatcggcggcgcctaccgcctgatcgacgtgcccatgagcaactgcatcaacagcggcatcagcaagatctacatcctgacccaggtgggtgagccgagccggcacgagcgtgccgtacccgtgtcgggtggccgggcgggagcgccctcgcgggagggcgatggcgcaggtgggctcgtacaggccctcttgctacgccggccgcggcaacacggcaaccgaacacgggaatgtgacctaccacttgcccccacatccgcaccgcgctgccctgcccacacatctgtgccacaccacacctcacctcaccttcgcaccgtcactcccaaccgccccacgccacccccgccacatttcctccctgcagttcaactcgacctccctgaaccgccacctgggtcgcgcctacaacatgggcagcggcgtgcgcttcggcggcgacggctttgtggaggtgctggcggccacccagacgcccaccgacaaggagtggttc

SEQ ID NO: 68 - DNA, 1320 bp, Synecocystis
  misc_feature (1)..(1320): glgC encoding polynucleotide sequence
gtgtgttgttggcaatcgagaggtctgcttgtgaaacgtgtcttagcgattatcctgggcggtggggccgggacccgcctctatcctttaaccaaactcagagccaaacccgcagttcccttggccggaaagtatcgcctcatcgatattcccgtcagtaattgcatcaactcagaaatcgttaaaatttacgtccttacccagtttaattccgcctcccttaaccgtcacatcagccgggcctataatttttccggcttccaagaaggatttgtggaagtcctcgccgcccaacaaaccaaagataatcctgattggtttcagggcactgctgatgcggtacggcaatacctctggttgtttagggaatgggacgtagatgaatatcttattctgtccggcgaccatctctaccgcatggattacgcccaatttgttaaaagacaccgggaaaccaatgccgacataaccctttccgttgtgcccgtggatgacagaaaggcacccgagctgggcttaatgaaaatcgacgcccagggcagaattactgacttttctgaaaagccccagggggaagccctccgggccatgcaggtggacaccagcgttttgggcctaagtgcggagaaggctaagcttaatccttacattgcctccatgggcatttacgttttcaagaaggaagtattgcacaacctcctggaaaaatatgaaggggcaacggactttggcaaagaaatcattcctgattcagccagtgatcacaatctgcaagcctatctctttgatgactattgggaagacattggtaccattgaagccttctatgaggctaatttagccctgaccaaacaacctagtcccgactttagtttttataacgaaaaagcccccatctataccaggggtcgttatcttccccccaccaaaatgttgaattccaccgtgacggaatccatgatcggggaaggttgcatgattaagcaatgtcgcatccaccactcagttttaggcattcgcagtcgcattgaatctgattgcaccattgaggatactttggtgatgggcaatgatttctacgaatcttcatcagaacgagacaccctcaaagcccggggggaaattgccgctggcataggttccggcaccactatccgccgagccatcatcgacaaaaatgcccgcatcggcaaaaacgtcatgattgtcaacaaggaaaatgtccaggaggctaaccgggaagagttaggtttttacatccgcaatggcatcgtagtagtgattaaaaatgtcacgatcgccgacggcacggtaatctag

SEQ ID NO: 69 - DNA, 1422 bp, Bacillus subtilis
  misc_feature (1)..(1422): SacB encoding polynucleotide sequence
atgaacatcaaaaaatttgcaaaacgagccacagttctaactttcacgactgcacttctggcagggggagcgactcaagccttcgcgaaagaaaatacccaaaaaccttacaaagaaacgtacggcgtctctcacatcacacgccatgatatgctgcagatccctaaacagcagcaaagtgaaaaataccaagtgcctcaattcgaccaatcaacaattaaaaatatcgagtccgcaaaaggactggatgtgtgggacagctggccgctccaaaacgctgacggaacagtagctgaatacaacggctatcacgtcgtgtttgctcttgctggaagcccgaaagacgctgatgacacatccatctacatgttttatcaaaaagtcggcgacaactcgatcgacagctggaaaaacgcgggccgtgtctttaaagacagcgataagttcgacgccaacgatgaaatcctgaaagaacagacacaagaatggtccggttctgcaacctttacatctgacggaaaaatccgtttattctacactgacttttccggtacacattacggcaaacaaagcctgacaacggcgcaggtaaatgtgtcaaaatctgatgacacgctcaagatcaacggagtggaagatcataaaacgatttttgacggtgacggaaaaacatatcaaaacgttcagcagtttatcgatgaagggaactatacatccggcgacaaccatacgctgagagaccctcactacgttgaagacaaaggccataaataccttgtattcgaagccaacactggaacagataacggataccaaggcgaagaatctttatttaacaaagcgtactacggcggcagcacaaacttcttccgtaaagaaagtcagaagcttcagcaaagtgctaaaaaacgcgatgctgaattagcgaatggcgccctcggtatggtagagttaaacgatgattacacattgaaaaaagtcatgaagccgctgattacttcaaatacggtaacagatgaaatcgagcgcgcgaatgttttcaaaatgaacggcaaatggtacctgttcactgattcacgcggttcaaaaatgacgatcgacggtattaactcaaacgatatttacatgcttggttatgtatcaaactctttaacaggtccttacaagccgctgaacaaaactggtcttgtactgcaaatgggtcttgaccctaacgatgtaacgttcacttactctcacttcgcagtgccgcaagccaaaggcaacaatgtcgtgatcacaagctacatgacaaacagaggcttctttgaggataaaaaggcaacatttgcgccaagcttcttaatgaacatcaaaggcaagaaaacatccgttgttaaaaacagcatccttgaacaaggacagcttacggttaacaactaa